(19) United States
(12) Patent Application Publication
(10) Pub. No.: US 2013/0143902 A1
(43) Pub. Date: Jun. 6, 2013

(54) COMPOSITIONS AND METHODS OF TREATING A PROLIFERATIVE DISEASE WITH A QUINAZOLINONE DERIVATIVE

(71) Applicant: Gilead Calistoga LLC, Foster City, CA (US)

(72) Inventors: Jerry B. Evarts, Redmond, WA (US); Heather Webb, Seattle, WA (US); Brian Lannutti, Seattle, WA (US)

(73) Assignee: GILEAD CALISTOGA LLC, Foster City, CA (US)

(21) Appl. No.: 13/691,524

(22) Filed: Nov. 30, 2012

Related U.S. Application Data
(60) Provisional application No. 61/566,572, filed on Dec. 2, 2011.

(51) Int. Cl. C07D 473/34; A61K 45/00; A61K 31/522

(52) U.S. Cl. CPC C07D 473/34; A61K 31/522; A61K 45/00
USPC 514/263.21; 544/277

(57) ABSTRACT

Provided are methods that relate to a novel therapeutic strategy for the treatment of cancers. In particular, the method comprises administration of Compound A,

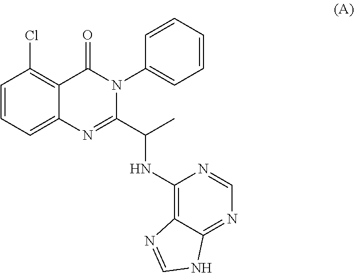 [SMILES: CC(Nc1ncnc2[nH]cnc12)c1nc2cccc(Cl)c2c(=O)n1-c1ccccc1]

or a pharmaceutically acceptable salt thereof, or a pharmaceutical composition comprising such compound admixed with at least one pharmaceutically acceptable excipient.

### CROSS-REFERENCE TO RELATED APPLICATIONS

This application claims the benefit of U.S. provisional patent application Ser. No. 61/566,572, filed Dec. 2, 2011, the entire disclosure of which is incorporated herein by reference.

### FIELD

The present application is in the field of therapeutics and medicinal chemistry. In particular, the present application concerns methods of treatment of cancer that include administration of certain quinazolinone derivatives.

### BACKGROUND

Cell signaling via 3′-phosphorylated phosphoinositides has been implicated in a variety of cellular processes, e.g., malignant transformation, growth factor signaling, inflammation, and immunity. The enzyme responsible for generating these phosphorylated signaling products, phosphatidylinositol 3-kinase (PI 3-kinase; PI3K), was originally identified as an activity associated with viral oncoproteins and growth factor receptor tyrosine kinases that phosphorylates phosphatidylinositol (PI) and its phosphorylated derivatives at the 3′-hydroxyl of the inositol ring.

PI 3-kinase activation is believed to be involved in a range of cellular responses including cell growth, differentiation, and apoptosis. In some instances, PI3K participates in cellular pathways involved in hematological malignancy and solid tumor activation. For example, PI3K participates in a cellular pathway that has been implicated in the process of oncogenic transformation and in promoting the growth, proliferation, and survival of various types of cancers, such as T-cell acute lymphoblastic leukemia.

The initial purification and molecular cloning of PI3-kinase revealed that it was a heterodimer consisting of p85 and p110 subunits. Four Class I PI3Ks have been identified and designated as PI3K α, β, δ, and γ isomers. Each isomer consists of a distinct p110 catalytic subunit and a regulatory subunit. Three catalytic subunits, p110α, p110β and p110δ, each interact with the same regulatory subunit, p85; whereas p110γ interacts with a distinct regulatory subunit, p101. The expression of each PI3K isoform in human cells and tissues are also distinct.

Identification of the p110δ isoform of PI-3-kinase is described in Chantry et al., J. Biol. Chem., 272:19236-41 (1997). It was observed that the human PI3K p110δ isoform was expressed in a tissue-restricted fashion; for example, PI3K p110δ expressed at high levels in lymphocytes and lymphoid tissues. This suggests that PI3Kδ might play a role in the PI3-kinase-mediated signaling in the immune system. The p110β isoform of PI3K may also play a role in the PI3K-mediated signaling in certain cancers.

Unexpected effects on PI3K isomers have been found in the compounds disclosed herein.

### SUMMARY

The present application discloses compounds, compositions and methods related to treating cancer or a condition related to PI3K-mediated disorders. Provided is a compound having the structure of Compound A

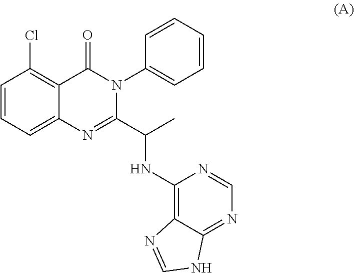 [SMILES: CC(Nc1ncnc2[nH]cnc12)c1nc2cccc(Cl)c2c(=O)n1-c1ccccc1]

or a pharmaceutically acceptable salt thereof. Provided are also all stereoisomeric forms, individual diastereoisomers and enantiomers, as well as racemic and non-racemic mixtures of Compound A or a pharmaceutically acceptable salt thereof.

In specific embodiments, the compound is the (S)-enantiomer, having the structure of Compound A(S):

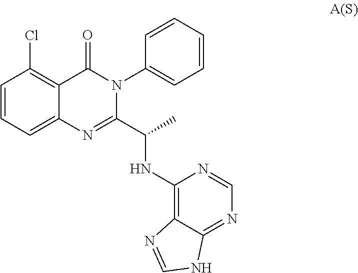 [SMILES: C[C@H](Nc1ncnc2[nH]cnc12)c1nc2cccc(Cl)c2c(=O)n1-c1ccccc1]

or a pharmaceutically acceptable salt thereof.

Provided is also a composition comprising a compound of any of the foregoing embodiments of Compound A, and at least one pharmaceutically acceptable excipient. In particular embodiments, the composition comprises a therapeutically effective amount of Compound A for the treatment of cancer in a patient.

In some embodiments, the compound is a racemic mixture of the (R)- and (S)-enantiomers of Compound A. In other embodiments, the compound is optically active. In specific embodiments, the (S)-enantiomer of Compound A, having the structure of Compound A(S):

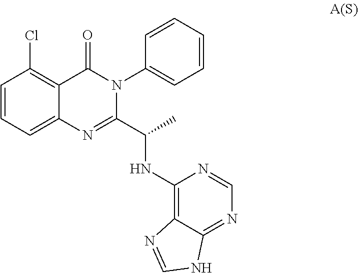 [SMILES: C[C@H](Nc1ncnc2[nH]cnc12)c1nc2cccc(Cl)c2c(=O)n1-c1ccccc1]

or a pharmaceutically acceptable salt thereof, is present in excess of Compound A(R)

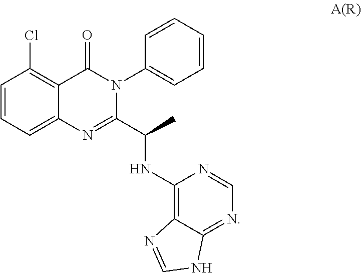 [SMILES: C[C@@H](Nc1ncnc2[nH]cnc12)c1nc2cccc(Cl)c2c(=O)n1-c1ccccc1]

In further embodiments, the compound is substantially free of Compound A(R). In some embodiments, the (S)-enantiomer of Compound A predominates over the (R)-enantiomer of Compound A by a molar ratio of at least 9:1, at least 19:1, at least 40:1, at least 80:1, at least 160:1, or at least 320:1.

The compound can also be described by its enantiomeric excess (e.e.). For instance, a compound with 95% (S)-isomer and 5% (R)-isomer will have an e.e. of 90%. In some embodiments, the compound has an e.e. of at least 60%, 75%, 80%, 85%, 90%, 95%, 98% or 99%. In some of the foregoing embodiments, the compound is enantiomerically-enriched in the (S)-isomer of Compound A.

Provided is also a method of treating cancer or a condition related to PI3K-mediated disorders. In certain embodiments, the method of treating a PI3K-mediated cancer comprises administering to a patient in need there of an effective amount of any of the foregoing compounds or compositions.

In certain embodiments, the cancer is a hematologic malignancy. In particular embodiments, the hematologic malignancy is leukemia or lymphoma. In specific embodiments, the hematologic malignancy is leukemia, wherein leukemia is selected from the group consisting of acute lymphocytic leukemia (ALL), acute myeloid leukemia (AML), chronic lymphocytic leukemia (CLL), and small lymphocytic lymphoma (SLL). In one embodiment, the cancer is T-cell acute lymphoblastic leukemia (T-ALL). In other specific embodiments, the hematologic malignancy is lymphoma, wherein lymphoma is selected from the group consisting of multiple myeloma (MM), non-Hodgkin's lymphoma (NHL), mantle cell lymphoma (MCL), follicular lymphoma, Waldestrom's macroglobulinemia (WM), T-cell lymphoma, B-cell lymphoma, and diffuse large B-cell lymphoma (DLBCL).

In other embodiments, the cancer is a solid tumor. In particular embodiments, the solid tumor is selected from the group consisting of pancreatic cancer, bladder cancer, colorectal cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancers, CNS cancers, brain tumors (e.g., glioma, anaplastic oligodendroglioma, adult glioblastoma multiforme, and adult anaplastic astrocytoma), bone cancer, and soft tissue sarcoma. In some embodiments, the solid tumor is selected from non-small cell lung cancer, small-cell lung cancer, colon cancer, CNS cancer, melanoma, ovarian cancer, renal cancer, prostate cancer and breast cancer.

In some of the foregoing embodiments, the patient's cancer is refractory to antitumor treatment or in relapse after antitumor treatment. In an alternative embodiment, the subject has not received prior antitumor treatment.

Specific embodiments provide a method of treating a condition in a patient, wherein the condition is cancer, comprising administering to the patient Compound A

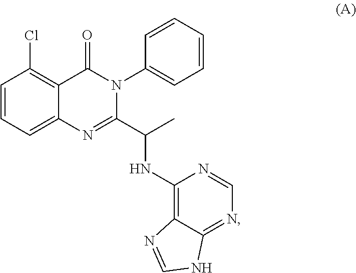 [SMILES: CC(Nc1ncnc2[nH]cnc12)c1nc2cccc(Cl)c2c(=O)n1-c1ccccc1]

or a pharmaceutically acceptable salt thereof, including all stereoisomeric forms, individual diastereoisomers and enantiomers, as well as racemic and non-racemic mixtures of Compound A or a pharmaceutically acceptable salt thereof.

Specific embodiments provide a method of treating a condition in a patient, wherein the condition is cancer, comprising administering to the patient a pharmaceutical composition comprising Compound A or a pharmaceutically acceptable salt thereof, optionally admixed with at least one pharmaceutically acceptable excipient. In particular embodiments, the composition comprises a therapeutically effective amount of a compound of any of the foregoing embodiments of Compound A or a pharmaceutically acceptable salt thereof for the treatment of cancer in a patient.

In some embodiments, the composition comprises a racemic mixture of Compound A. In a specific embodiment, the composition comprises the (S)-enantiomer of Compound A, having the structure of Compound A(S):

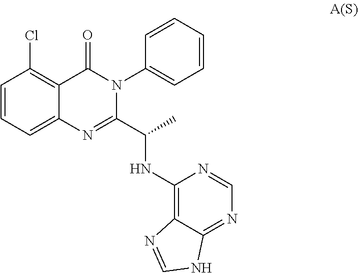 [SMILES: C[C@H](Nc1ncnc2[nH]cnc12)c1nc2cccc(Cl)c2c(=O)n1-c1ccccc1]

or a pharmaceutically acceptable salt thereof, wherein Compound A(S) is present in excess of the (R)-enantiomer of Compound A, having the structure of Compound A(R)

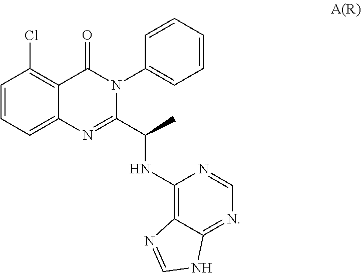 [SMILES: C[C@@H](Nc1ncnc2[nH]cnc12)c1nc2cccc(Cl)c2c(=O)n1-c1ccccc1]

In specific embodiments, the composition is substantially free of the (R)-enantiomer of Compound A.

In some of the foregoing embodiments, Compound A is administered at a dose of about 1 to 4,000 mg/day, about 2,000 to 4,000 mg/day, about 1 to 2,000 mg/day, about 1 to 1,000 mg/day, about 10 to 500 mg/day, about 20 to 500 mg/day, about 50 to 300 mg/day, about 75 to 200 mg/day, or about 15-150 mg/day. In other embodiments, Compound A is administered at a dose of about 1 to 150 mg twice per day. In yet other embodiments, Compound A is administered at least twice daily. In certain embodiments, Compound A is administered intermittently or in intervals. Depending on the treatment and the patient's condition, the interval may range from one, two, three, four, five, six and seven days. In one example, Compound A is administered for at least 21 days, and is then discontinued for at least 7 days. In another example, Compound A is administered for about 21 days, and is then discontinued for about 7 days.

In some of the foregoing embodiments, the method further comprises reducing the level of PI3Kδ, PI3Kγ, and/or PI3Kβ activity in the patient in need thereof. In certain embodiments, the method further comprises reducing the level of PI3Kδ and PI3Kγ activity in the patient in need thereof. In some of the foregoing embodiments, the method further comprises reducing the level of PI3Kδ and PI3Kβ in the patient in need thereof. In some of the foregoing embodiments, the method further comprises reducing the level of PI3Kδ, PI3Kγ, and PI3Kβ activity in the patient in need thereof. In some of foregoing embodiment, the method further comprises the PI3Kα-sparing activity in the patient in need thereof.

In some of the foregoing embodiments, the method further comprises administering to a patient, in addition to Compound A, a therapeutically effective amount of at least one therapeutic agent selected to treat the cancer or autoimmune disease in the patient. In some embodiments, the therapeutic agent is selected from the following group consisting of Bortezomib (VELCADE®), Carfilzomib (PR-171), PR-047, disulfuram, lactacystin, PS-519, eponemycin, epoxomycin, aclacinomycin, CEP-1612, MG-132, CVT-63417, PS-341, vinyl sulfone tripeptide inhibitors, ritonavir, PI-083, (+/−)-7-methylomuralide, (−)-7-methylomuralide, Perifosine, Rituximab, Sildenafil citrate (VIAGRA®), CC-5103, Thalidomide, Epratuzumab (hLL2-anti-CD22 humanized antibody), Simvastatin, Enzastaurin, Campath-1H, Dexamethasone, DT PACE, oblimersen, antineoplaston A10, antineoplason AS2-1, alemtuzumab, beta alethine, cyclophosphamide, doxorubicin hydrochloride, PEGylated liposomal doxorubicin hydrochloride, prednisone, prednisolone, cladribine, vincristine sulfate, fludarabine, filgrastim, melphalan, recombinant interferon alfa, carmustine, cisplatin, metformin, rosiglitazone, pioglitazone, cyclophosphamide, cytarabine, etoposide, melphalan, dolastatin 10, indium In 111 monoclonal antibody MN-14, yttrium Y 90 humanized epratuzumab, anti-thymocyte globulin, busulfan, cyclosporine, methotrexate, mycophenolate mofetil, therapeutic allogeneic lymphocytes, Yttrium Y 90 ibritumomab tiuxetan, sirolimus, tacrolimus, carboplatin, thiotepa, paclitaxel, aldesleukin, recombinant interferon alfa, docetaxel, ifosfamide, mesna, recombinant interleukin-12, recombinant interleukin-11, Bcl-2 family protein inhibitor ABT-263, denileukin diftitox, tanespimycin, everolimus, pegfilgrastim, vorinostat, alvocidib, recombinant flt3 ligand, recombinant human thrombopoietin, lymphokine-activated killer cells, amifostine trihydrate, aminocamptothecin, irinotecan hydrochloride, caspofungin acetate, clofarabine, epoetin alfa, nelarabine, pentostatin, sargramostim, vinorelbine ditartrate, WT-1 analog peptide vaccine, WT1 126-134 peptide vaccine, fenretinide, ixabepilone, oxaliplatin, monoclonal antibody CD19, monoclonal antibody CD20, omega-3 fatty acids, mitoxantrone hydrochloride, octreotide acetate, tositumomab and iodine I131 tositumomab, motexafin gadolinium, arsenic trioxide, tipifarnib, autologous human tumor-derived HSPPC-96, veltuzumab, bryostatin 1, anti-CD20 monoclonal antibodies, chlorambucil, pentostatin, lumiliximab, apolizumab, Anti-CD40, ofatumumab, bendamustine, and a combination thereof.

In other embodiments, the therapeutic agent is a proteasome inhibitor.

Another aspect provides a method of treating a T-cell malignancy, comprising selective activity of inhibiting phosphoinositide 3-kinase (PI3K) isoform activities in T-cells, thereby treating the T-cell malignancy. In some embodiments, the method comprises administering a compound of the present application. In one embodiment, the T-cell malignancy is T-cell acute lymphoblastic leukemia (T-ALL). In another embodiment, the T-cell malignancy is a T-cell lymphoma.

In some embodiments of the method of treating the T-cell malignancy, selectively inhibiting comprises administering at least one selective inhibitor in an amount effective to inhibit p110δ and p110γ in T-cells. In certain embodiments, at least one of the selective inhibitors further inhibits p110β. In one embodiment, the selectively inhibiting is in vitro. In another embodiment, the selectively inhibiting is in vivo.

In some embodiments of the method of treating the T-cell malignancy, at least one of the selective inhibitors has an in vitro PI3Kγ IC₅₀ to PI3Kδ IC₅₀ ratio of between 0.05 and 500. In certain embodiments, at least one of the selective inhibitors has an in vitro PI3Kγ IC₅₀ to PI3Kδ IC₅₀ ratio of between 200 and 400. In certain embodiments, at least one of the selective inhibitors has an in vitro PI3Kγ EC₅₀ to PI3Kδ EC₅₀ ratio of between 0.05 and 350. In certain embodiments, at least one of the selective inhibitors has an in vitro PI3Kγ EC₅₀ to PI3Kδ EC₅₀ ratio of between 200 and 300.

In some embodiments of the method of treating the T-cell malignancy, at least one of the selective inhibitors is a compound having formula I

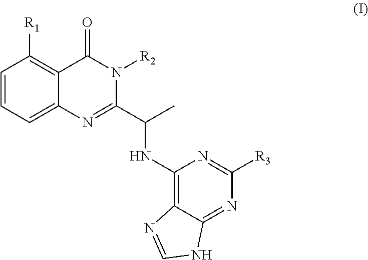

or a pharmaceutically acceptable salt thereof,

wherein R₁ is hydrogen, halo, or C₁₋₆ alkyl;

wherein R₂ is aryl or heteroaryl; and

wherein R₃ is hydrogen, halo, or amino.

In certain embodiments of the method of treating the T-cell malignancy, wherein at least one of the selective inhibitor is 2-(1-(2-amino-9H-purin-6-ylamino)ethyl)-5-methyl-3-o-tolylquinazolin-4(3H)-one, or 2-(1-(9H-purin-6-ylamino)ethyl)-5-chloro-3-phenylquinazolin-4(3H)-one, or a pharmaceutically acceptable salt thereof, including all stereoisomeric forms, enantiomers thereof as well as racemic and non-racemic mixtures thereof.

### BRIEF DESCRIPTION OF THE FIGURES

FIG. 1A. Kaplan-Meyer survival curves showing the role of PI3Kγ and PI3Kδ in the development of PTEN-null T-ALL. FIG. 1B. Flow cytometric profiles of peripheral blood from diseased mice lacking PI3K p110γ or p110δ in the absence of PTEN in T-cell progenitors. Forward scatter (FSC) and Ki67 staining indicate cell size and proliferation, respectively, and Thy 1.2 expression identifies T-lineage cells. FIG. 1C Immunoblots depicting PI3K p110γ, PI3K p110δ, and PTEN expression as well as Akt/PKB activation state (phosphorylation of Ser473 of Akt) in thymic lysates.

FIG. 2A. Hematoxylin and eosin staining and flow cytometric profiles of thymi derived from 6-week old mice lacking both PI3K p110γ and p110δ in the presence or absence of PTEN. The panels are representative of data from five animals in each group. Cell counts represent the means±s.d. FIG. 2B Immunoblots of Akt/PKB phosphorylation and PTEN in thymic lysates of transgenic mice and wild-type (WT) control mice. FIG. 2C. The number of white blood cell count (WBC) and T-cell subsets in the peripheral blood. Data represent the mean±s.d. *P>0.05, **P<0.01. FIG. 2D. Hematoxylin and eosin staining of spleen and peripheral lymph nodes. FIG. 2E. Flow cytometry analyses of blood and spleen in PTEN^{fl/fl} PI3Kγ^{ko}/PI3Kδ^{ko} mice (n=5 mice per genotype). T-cells were identified by immunoperoxidase detection of CD3. Bars correspond to 200 μm in secondary lymphoid organs and to 500 μm in thymi.

FIG. 3. Flow cytometric profiles of diseased Lck/PTEN^{fl/fl} mice administered with Compound A of 30 mg/kg BID at Day 0, 4, and 7.

FIGS. 4A-D. Kaplan-Meyer survival curve, peripheral blood smears, and flow cytometric profiles for diseased Lck/PTEN^{fl/fl} treated with Compound B for a period of 7 d. P<0.001. Numbers represent the initial WBC (×10⁶) for each animal prior to treatment. FIG. 4E. Kaplan-Meyer survival curve, peripheral blood smears, and flow cytometric profiles for diseased Lck/PTEN^{fl/fl} PI3Kγ^{ko} mice treated with Compound E for a period of 7 days. An untreated wild type animal was shown for comparison in FIG. 4A. FIG. 4F. Bioluminescent images and corresponding flow cytometric profiles of diseased Lck/PTEN^{fl/fl} animals treated with Compound B. FIG. 4G. Weights of thymi, liver, spleen, and kidneys harvested from diseased Lck/PTEN^{fl/fl mice} 7 days post-treatment with either Compound B or vehicle control (n=5, *P<0.01). Peripheral blood counts (WBC, right axis) represent the mean±s.d. prior to treatment.

FIG. 5A-B. Proliferation of CCRF-CEM cells treated with Compound B or vehicle control. FIG. 5B. Survival of CCRF-CEM cells treated with Compound B or vehicle control. FIG. 5C. Proliferation of CCRF-CEM cells without p110γ expression when treated with Compound E. FIG. 5D. Survival of CCRF-CEM cells without p110γ expression when treated with Compound E. Data represent the mean±s.d. of triplicate experiments. *P<0.01, **P<0.001. FIG. 5E. Effect of Compound B on signaling pathways downstream of PI3Kγ and PI3Kδ in CCRF-CEM cells. FIG. 5F. Activation of the pro-apoptotic pathway in CCRF-CEM cells treated with Compound B. FIG. 5G. Bioluminescence images (upper panel) and quantification of tumor mass changes (lower panel) in mice with subcutaneous CCRF-CEM xenografts treated with DMSO vehicle control or Compound B for 4 days (n=7). FIG. 5H. Kaplan-Meyer survival curve of mice treated with vehicle control or Compound B for 7 days in a systemic CCRF-CEM xenograft model (P<0.01; n=7 per group).

FIG. 6A. Viability of tumors treated with increasing concentrations of Compound B for 72 hours. Percent viability indicates the proportion of live-gated cells in the treated populations relative to its untreated counterpart. FIG. 6B Immunoblots analysis of p110 and PTEN expression as well as phosphorylation state of Akt/PKB in primary T-ALL tumors. FIG. 6C. Effect of Compound B on the Akt/PKB phosphorylation after 6 hours of treatment. Densitometry was performed on bands from immunoblots. The P-Akt signal was normalized to total Akt.

FIG. 7A. Viability of T-ALL tumors treated with Compound A or a vehicle control. FIG. 7B Immunoblot analysis of p110 and PTEN expression in primary T-ALL tumors treated with Compound A.

FIG. 8A. Proliferation of T-cells cultured with Compounds A, B or DMSO vehicle control. FIG. 8B. Survival of T-cells cultured with Compounds A, B or DMSO vehicle control.

### DETAILED DESCRIPTION

Unless otherwise defined, all terms of art, notations and other scientific terms or terminology used herein are intended to have the meanings commonly understood by those of skill in the art to which this present disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted.

The discussion of the general methods given herein is intended for illustrative purposes only. Other alternative methods and embodiments will be apparent to those of skill in the art upon review of this disclosure.

A group of items linked with the conjunction “or” should not be read as requiring mutual exclusivity among that group, but rather should also be read as “and/or” unless expressly stated otherwise. Although items, elements, or components of the present disclosure may be described or claimed in the singular, the plural is contemplated to be within the scope thereof unless limitation to the singular is explicitly stated.

### Compound A

Provided are novel methods to treat cancer or a condition related to PI3K-mediated disorders using Compound A. One aspect provides a compound having the structure of Compound A

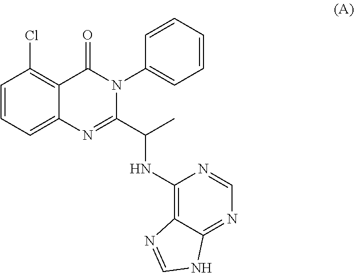 [SMILES: CC(Nc1ncnc2[nH]cnc12)c1nc2cccc(Cl)c2c(=O)n1-c1ccccc1]

or a pharmaceutically acceptable salt thereof, including all stereoisomeric forms, individual diastereoisomers and enantiomers as well as racemic and non-racemic mixtures of Compound A or a pharmaceutically acceptable salt thereof. Another aspect provides a pharmaceutical composition comprising Compound A or a pharmaceutically acceptable salt thereof, optionally admixed with at least one pharmaceutically acceptable excipient.

In specific embodiments, the compound is the (S)-enantiomer, having the structure of Compound A(S):

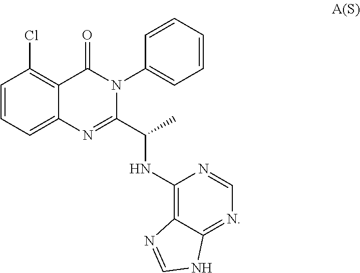 [SMILES: C[C@H](Nc1ncnc2[nH]cnc12)c1nc2cccc(Cl)c2c(=O)n1-c1ccccc1]

Provided is also Compound A in which from 1 to n hydrogens attached to a carbon atom may be replaced by deuterium, in which n is the number of hydrogens in the molecule. Such compounds exhibit may increase resistance to metabolism, and thus may be useful for increasing the half life of Compound A when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogens have been replaced by deuterium.

Compositions comprising Compound A may include racemic mixtures or mixtures containing an enantiomeric excess of one enantiomer or single diastereomers or diastereomeric mixtures. All such isomeric forms of these compounds are expressly included herein the same as if each and every isomeric form were specifically and individually listed.

Compound A and compositions thereof for use in the methods described herein may be optically active. Compound A has a single chiral center in the noncyclic linking group between the quinazolinone moiety and the purine moiety. In some embodiments, the preferred enantiomer of Compound A is the (S)-enantiomer depicted above. Optically active forms of Compound A may include predominantly the (S)-enantiomer, although it may also include the (R)-enantiomer of Compound A as a minor component. For clarity, where a dosage of Compound A is described herein, the dosage refers to the weight of Compound A, including each enantiomer that may be present. Thus, a dosage of 100 mg of Compound A as used herein, for example, refers to the weight of the mixture of enantiomers rather than the weight of the (S)-enantiomer specifically. It could, for example, refer to 100 mg of a 9:1 mixture of (S)- and (R)-enantiomers, which would contain about 90 mg of the (S)-enantiomer, or to 100 mg of a 19:1 mixture of (S)- and (R)-enantiomers, which would contain about 95 mg of the (S)-enantiomer.

Compound A may be synthesized in optically active form, or it may be prepared in racemic form (containing equal amounts of (R)- and (S)-isomers), and then the isomers may be separated. Scheme 1 depicts a chiral synthesis of Compound A that provides the (S)-enantiomer in very high optical purity. In some embodiments, the enantiomeric (R)-isomer of Compound A may be excluded. In other embodiments, the methods may be practiced with mixtures of (R)- and (S)-isomers. In yet other embodiments, the methods may be practiced with mixtures of (R)- and (S)-isomers, in which the (S)-isomer is the major component of the mixture. In embodiments where the (S)-isomer is the major component of the mixture, such mixture may contain no more than about 10% of the (R)-isomer, meaning the ratio of (S)- to (R)-isomers is at least about 9:1, and in other embodiments, less than 5% of the (R)-isomer, meaning the ratio of (S)- to (R)-enantiomers is at least about 19:1. In some embodiments, the (S)-enantiomer predominates over the (R)-enantiomer by a molar ratio of at least 40:1, at least 80:1, at least 160:1, or at least 320:1.

Compound A can also be described by its enantiomeric excess (e.e.). For instance, a compound characterized by 95% (S)-isomer and 5% (R)-isomer will have an e.e. of 90%. In some embodiments, the Compound A has an e.e. of at least 60%, at least 75%, at least 80%, at least 90%, at least 95%, at least 98%, or at least 99%.

In some of the foregoing embodiments, the compound is enantiomerically-enriched in the (S)-isomer of Compound A. In certain embodiments, the compound may be enriched with the (S)-enantiomer shown here:

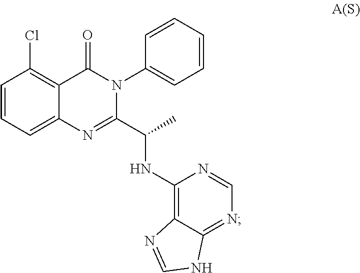 [SMILES: C[C@H](Nc1ncnc2[nH]cnc12)c1nc2cccc(Cl)c2c(=O)n1-c1ccccc1]

and preferably, is at least 90% (S)-enantiomer of Compound A, containing no more than about 10% of the enantiomeric (R)-enantiomer of Compound A.

In certain embodiments, Compound A is primarily composed of the (S)-enantiomer of Compound A, wherein this isomer comprises at least 66-95%, or about 85-99% of the (S)-enantiomer, in excess over any (R)-enantiomer present. In certain embodiments, Compound A is at least 95% of the (S)-enantiomer. In one embodiment, Compound A is 100% of the (S)-enantiomer. In the additional embodiment, Compound A is at least 99% of the (S)-enantiomer, with less than 1% of the (R)-enantiomer.

The compounds depicted herein may be present as salts even if salts are not depicted. In some embodiments, the salts of the compounds of the invention are pharmaceutically acceptable salts.

### Methods of Treatment

The methods described herein are useful to treat cancer or a condition related to PI3K-mediated disorders, such as a hematological malignancy and/or solid tumor. “Treating” as used herein refers to inhibiting a disorder (such as, for example, arresting its development), relieving the disorder (such as, for example, causing its regression), or ameliorating the disorder (such as, for example, reducing the severity of at least one of the symptoms associated with the disorder). “Disorder” is intended to encompass medical disorders, diseases, conditions, syndromes, and the like, without limitation.

One aspect provides methods of using Compound A or compositions thereof to inhibit the growth or proliferation of cancer cells of hematopoietic origin, such as cancer cells of lymphoid origin. Cancers amenable to treatment using the methods described herein include, without limitation, lymphomas, e.g., malignant neoplasms of lymphoid and reticuloendothelial tissues, such as multiple myeloma (MM), non-Hodgkin's lymphoma (NHL), mantle cell lymphoma (MCL), Waldenstrom's macroglobulinemia (WM) T-cell lymphoma, B-cell lymphoma, diffuse large B-cell lymphoma (DLBCL) and the like; as well as leukemias such as acute lymphocytic leukemia (ALL), acute myeloid leukemia (AML), chronic lymphocytic leukemia (CLL), small lymphocytic lymphoma (SLL), and the like.

In certain embodiments, the cancer is a hematologic malignancy. In particular embodiments, the hematologic malignancy is leukemia or lymphoma. In specific embodiments, the hematologic malignancy is leukemia, wherein leukemia is selected from the group consisting of acute lymphocytic leukemia (ALL), acute myeloid leukemia (AML), chronic lymphocytic leukemia (CLL), and small lymphocytic lymphoma (SLL). In other specific embodiments, the hematologic malignancy is lymphoma, wherein lymphoma is selected from the group consisting of multiple myeloma (MM), non-Hodgkin's lymphoma (NHL), mantle cell lymphoma (MCL), follicular lymphoma, Waldestrom's macroglobulinemia (WM), T-cell lymphoma, B-cell lymphoma, diffuse large B-cell lymphoma (DLBCL), and T-cell acute lymphoblastic leukemia (T-ALL). In one embodiment, the cancer is T-cell acute lymphoblastic leukemia (T-ALL).

Another aspect includes methods of using Compound A or compositions thereof to treat a solid tumor, typically a non-hematopoietic carcinoma. In some embodiments, the cancer is a solid tumor selected from pancreatic cancer, bladder cancer, colorectal cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancers, CNS cancers, brain tumors (e.g., glioma, anaplastic oligodendroglioma, adult glioblastoma multiforme, and adult anaplastic astrocytoma), bone cancer, and soft tissue sarcoma. In some embodiments, the cancer is CNS cancer, renal cancer, prostate cancer, melanoma, ovarian cancer, breast cancer, colon cancer, and brain tumors (e.g., glioma tumors).

Another aspect includes methods of using Compound A or compositions thereof to treat a condition related to PI3K-mediated disorders such as inflammation or inflammatory disease. Inflammation is a localized, protective response elicited by injury or destruction of tissues, which serves to destroy, dilute or wall off (i.e., sequester) both the injurious agent and the injured tissue. Inflammation or inflammatory disease can be acute or chronic, and often involves the immune response. Inflammation typically results from a cascade of events that includes vasodilation accompanied by increased vascular permeability and exudation of fluid and plasma proteins. The disruption of vascular integrity precedes or coincides with an infiltration of inflammatory cells. Inflammatory mediators generated at the site of the initial lesion serve to recruit inflammatory cells to the site of injury. These mediators (chemokines such as IL-8, MCP-1, MIP-1, and RANTES, complement fragments and lipid mediators) have chemotactic activity for leukocytes and attract the inflammatory cells to the inflamed lesion. These chemotactic mediators, which cause circulating leukocytes to localize at the site of inflammation, require the cells to cross the vascular endothelium at a precise location. This leukocyte recruitment is accomplished by a process called cell adhesion. Inflammatory disease occurs when the normal discontinuation or attenuation of an inflammatory response does not occur or is incomplete.

The terms ‘inflammation’, ‘inflammatory disease’ or variant thereof are used to refer to any disease in which an excessive or unregulated inflammatory response leads to excessive inflammatory symptoms, host tissue damage, or loss of tissue function. This includes but not limited to autoimmune disease, allergic disease, arthritic disease, asthma, acne, dermatitis, hypersensitive, transplant rejection, and inflammatory bowel disease.

While not wishing to be bound by any theory, the efficacy of Compound A is believed to arise from its in vivo inhibition of PI3K isoforms other than PI3Kα or the PI3Kα-sparing activity. Based on its PI3Kα-sparing activity, Compound A may be suitable to therapeutically target certain cancers or conditions related to PI3K-mediated disorders. As demonstrated in the examples described herein, propagation of upstream signaling pathways critical for the development and/or survival of PTEN null T-ALL tumors rely mainly on PI3Kγ and PI3Kδ. Since both PI3Kγ and PI3Kδ are involved in the oncogenic process in T-cell progenitors in the absence of appropriate regulation, and can provide sufficient growth and survival signals necessary for tumor cell maintenance, selective PI3Kδ/γ inhibitors or compounds having PI3Kα-sparing activity can be therapeutically targeted for the treatment of T-cell malignancies, such as T-ALL.

As used herein, the term ‘the PI3Kα-sparing activity’ or variant thereof refers to compounds having selectivity activity of greater than 5-fold for PI3K isoforms β, δ, and γ over the PI3Kα isoform in cellular assays. For example, the compound having the PI3Kα-sparing activity reduces the activity of PI3Kδ and PI3Kγ response more than that of PI3Kα. In another example, the compound having the PI3Kα-sparing activity reduces the activity of PI3Kδ, PI3Kγ, and PI3Kβ response more than that of PI3Kα. The compound having the PI3Kα-sparing activity is a selective inhibitor to some PI3K isoforms β, δ, and γ over the PI3Kα isoform.

One example of such activity is the EC₅₀ values shown in Table 5. As seen in the cellular assay in Example 4, Compound A inhibits PI3Kδ response with an EC₅₀ of about 2.4 nM, PI3Kγ with an EC₅₀ of about 677 nM, and PI3Kβ with an EC₅₀ of about 270 nM, while showing much less activity on PI3Kα with an EC₅₀ of 6,000 nM. Also shown in the examples herein, Compound A has an unexpected effect as a potent and selective inhibitor having the PI3Kα-sparing activity.

As used herein, the term ‘potency’ or variant thereof refers to one compound has an increased levels of activity when compared to other compounds at a specific concentration. In one preferred embodiment, the potency is the PI3Kα-sparing activity exerted by the compound disclosed herein. By way of example, the potency of the compound is determined by the IC₅₀ value, which can be determined using commonly available methods; including in vitro enzyme assays or in vitro protein kinase assays. As understood by a person skilled in the art, a compound having a lower IC₅₀ value is more potent than a compound having higher IC₅₀ value. Also used herein, the term ‘selectivity’ or variant thereof refers to one compound has an increased level of activity on one isoform than other isoforms. In one preferred embodiment, the selectivity is the activity on some PI3K isoform and not other PI3K isoforms exerted by the compound disclosed herein. By way of example, the selectivity is determined using the EC₅₀ value, which can be determined using commonly available methods for cellular assays. As understood by a person skilled in the art, a compound having a lower EC₅₀ value is more selective than a compound having a higher EC₅₀ value. In one embodiment, the compound having the PI3Kα-sparing activity has an in vitro PI3Kγ IC₅₀ to PI3Kδ IC₅₀ ratio of between 0.05 and 500. In other embodiment, the compound having the PI3Kα-sparing activity has an in vitro PI3Kγ IC₅₀ to PI3Kδ IC₅₀ ratio of between 200 and 400. In some embodiment, the compound having the PI3Kα-sparing activity having an in vitro PI3Kγ EC₅₀ to PI3Kδ EC₅₀ ratio of between 0.05 and 350. In yet other embodiment, the compound having the PI3Kα-sparing activity has in vitro PI3Kγ EC₅₀ to PI3Kδ EC₅₀ ratio of between 200 and 300.

Also provided herein are methods of treating T-ALL by administering a compound having the PI3Kα-sparing activity. In some embodiments, Compound A, which has the PI3K-sparing activity, may be administered to treat T-ALL. Other compounds that may be administered as a compound having the PI3Kα-sparing activity to treat T-ALL may include, for example, Compounds B, C, D and E or a pharmaceutically acceptable salt thereof, or a pharmaceutical composition comprising the compound or a pharmaceutically acceptable salt thereof, optionally admixed with at least one pharmaceutically acceptable excipient.

In some of the foregoing embodiments, the method further comprises reducing the level of PI3Kδ and PI3Kγ activity in the patient. In some of the foregoing embodiments, the method further comprises reducing the level of PI3Kδ, PI3Kγ, and PI3Kβ activity in the patient.

In some embodiments, the subject for treatments described herein is one who has been diagnosed with at least one of the cancers described herein as treatable by the use of Compound A. In some embodiments, the subject has been diagnosed with a cancer or a condition related to PI3K-mediated disorders named herein, and has a cancer that has proven refractory to treatment with at least one conventional antitumor agent. Thus, in one embodiment, the treatments described herein are directed to patients who have received one or more than one such treatment and remain in need of more effective treatment. In some of the foregoing embodiments, the subject is a patient with a cancer that is refractory to antitumor treatment or in relapse after antitumor treatment.

### Dosing and Modes of Administration

Treatments of the methods described herein typically involve administration of Compound A to a subject in need of treatment on a daily basis for at least one week or more than one week. For example, Compound A is administered to a subject in need thereof on a daily basis for 2 to 4 weeks, for 3 to 4 weeks, or for 1 month or more. In some embodiments, Compound A may be administered in multiple doses each day, in order to maintain efficacious plasma levels over a prolonged period of time. Administration may be done in one dose per day, two doses per day, three doses per day, or four doses per day. Alternatively, Compound A can be administered intravenously at a rate that maintains an efficacious plasma level for a prolonged period of time.

The therapeutically effective amount can be determined by one of ordinary skill based on the subject's health, age, body weight, and condition. In some embodiments, the amount is normalized to the subject's body weight. For example, a dosage may be expressed as a number of milligrams of Compound A per kilogram of the subject's body weight (mg/kg). Dosages of between about 0.1 and 150 mg/kg are often appropriate, and in some embodiments, about 0.1 and 100 mg/kg are often appropriate, and in other embodiments a dosage of between 0.5 and 60 mg/kg is used. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

In other embodiments, the daily dosage may be described as a total amount of Compound A administered per dose or per day. Daily dosage of Compound A is typically between about 1 mg and 4,000 mg. In some embodiments, Compound A is administered at a dose of about 2,000 to 4,000 mg/day. In other embodiments, Compound A is administered at a dose of about 1 to 2,000 mg/day. In yet other embodiments, Compound A is administered at a dose of about 1 to 1,000 mg/day. In addition embodiments, Compound A is administered at a dose of about 10 to 500 mg/day. In other embodiment, Compound A is administered at a dose of about 20 to 500 mg/day. In other embodiments, Compound A is administered at a dose of about 50 to 300 mg/day. In yet another embodiments, Compound A is administered at a dose of about 75 to 200 mg/day. In other embodiment, Compound A is administered at a dose of about 15-150 mg/day.

When administered orally, the total daily dosage for a human subject is typically between 1 mg and 1,000 mg. In a particular embodiment, Compound A is administered at a dose of about 10-500 mg/day. In a particular embodiment, Compound A is administered at a dose of about 50-300 mg/day. In a particular embodiment, Compound A is administered at a dose of about 75-200 mg/day. In a particular embodiment, Compound A is administered at a dose of about 100-150 mg/day.

In a particular embodiment, Compound A is administered at a dose of about 1 to 150 mg per dose, and one to four doses are administered per day (e.g., QD dosing with about 1 to 150 mg, BID dosing with about 1 to 150 mg, or TID dosing with doses between about 1 to 150 mg, or QID dosing with doses between about 1 to 150 mg). In a preferred embodiment, a subject is treated with about 1 mg to 150 mg doses of Compound A once, twice, three, or four times per day. As used herein, the term QD refers to dosing once per day, BID refers to dosing twice per day, TID refers to dosing three times per day and QID refers to dosing four times per day.

Treatment with the compounds described herein is frequently continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles.

In a particular embodiment, the method comprises administering to the subject an initial daily dose of about 1 to 500 mg of Compound A and increasing the dose by increments until clinical efficacy is achieved. Increments of about 5, 10, 25, 50, or 100 mg can be used to increase the dose. The dosage can be increased daily, every other day, twice per week, or once per week.

In a particular embodiment, this method comprises continuing to treat the subject by administering Compound A at a dosage where clinical efficacy is achieved for a week or more, or reducing the dose by increments to a level at which safety and efficacy can be maintained. Safety can be monitored by conventional methods such as evaluating serum chemistry and complete blood count parameters. Efficacy can be monitored by conventional methods such as assessing tumor size or spreading (metastasis).

In a particular embodiment, the method comprises administering to the subject an initial daily dose of about 1 to 500 mg of Compound A and increasing the dose to a total dosage of about 50 to 400 mg per day over at least 6 days. Optionally, the dosage can be further increased to about 750 mg/day.

In a particular embodiment, Compound A is administered once daily. In another embodiment, Compound A is administered at least twice daily. In some embodiments Compound A is administered three times per day. In some embodiments, Compound A is administered four times per day, or more than four times per day.

In a particular embodiment, Compound A is administered at a rate selected to produce a concentration of compound in the blood between about 40 to 4,000 ng/mL, and maintaining such concentration during a period of about 4 to 12 hours following administration. In another particular embodiment, the dose size and frequency are selected to achieve a concentration of compound in the blood that is between about 75 to 2,000 ng/mL and maintain that concentration during a period of about 4 to 12 hours from the time of administration. In some embodiments, the dose size and frequency are selected to achieve a concentration of compound in the blood that is between about 100 to 1,000 ng/mL following administration. In some embodiments, the dose size and frequency are selected to achieve a concentration of compound in the blood that is between about 100 to 500 ng/mL over a period of about 12 to 24 hours from the time of administration. In some embodiments, the dose size and frequency are selected to achieve a Cₘₐₓ, plasma level of Compound A that is at least about 500 ng/mL and does not exceed about 10,000 ng/mL.

In certain embodiments, Compound A is administered orally, intravenously, transdermally, or by inhalation. Preferably, Compound A is administered orally. In some embodiments, Compound A is administered orally in a dose of about 1 mg, 3 mg, 15 mg, 20 mg, 25 mg, 30 mg, 40 mg, 50 mg, 60 mg, 75 mg, or 100 mg, 125 mg, 150 mg, 200 mg, or 300 mg per dose, and the dose may be administered at a frequency of once per day, twice per day, three times per day, or four times per day. In other embodiments, it is administered orally in a dose of about 15 mg, 20 mg, 25 mg, 30 mg, 40 mg, 50 mg, 60 mg, 75 mg, or 100 mg, 125 mg, or 150 mg per dose, and the dose may be administered at a frequency of once per day, twice per day, three times per day, or four times per day.

In a particular embodiment, the method comprises administering to a patient, in addition to Compound A, a therapeutically effective amount of at least one therapeutic agent selected to treat the cancer in the patient. In certain embodiments, Compound A may be combined with one or more other active therapeutic agents in a unitary dosage form for simultaneous or sequential administration to a patient. The combination therapy may be administered as a simultaneous or sequential regimen. When administered sequentially, the combination may be administered in two or more administrations.

In one embodiment, co-administration of Compound A with one or more other active therapeutic agents generally refers to simultaneous or sequential administration of Compound A and one or more other active therapeutic agents, such that therapeutically effective amounts of Compound A and one or more other active therapeutic agents are both present in the body of the patient. In an alternative embodiment, Compound A and therapeutic agent(s) are not necessarily both present in the body of the patient but the particular dosing schedule Compound A and therapeutic agents results in synergistic effects.

Co-administration includes administration of unit dosages of Compound A before or after administration of unit dosages of one or more other active therapeutic agents; for example, administration of Compound A within seconds, minutes, hours or days of the administration of one or more other active therapeutic agents. For example, a unit dose of Compound A can be administered first, followed within seconds, minutes, hour or days by administration of a unit dose of one or more other active therapeutic agents. Alternatively, a unit dose of one or more other therapeutic agents can be administered first, followed by administration of a unit dose of Compound A within seconds, minutes, hours or days. In some cases, it may be desirable to administer a unit dose of Compound A first, followed, after a period of hours (e.g., 1 to 12 hours), by administration of a unit dose of one or more other active therapeutic agents. In other cases, it may be desirable to administer a unit dose of one or more other active therapeutic agents first, followed, after a period of hours (e.g., 1 to 12 hours), by administration of a unit dose of Compound A. In some cases, it may be desirable to administer a unit dose of Compound A first, followed, after a period of days (e.g., 1 to 14 days), by administration of a unit dose of one or more other active therapeutic agents. In other cases, it may be desirable to administer a unit dose of one or more other active therapeutic agents first, followed, after a period of days (e.g., 1 to 14 days), by administration of a unit dose of Compound A. In some embodiments, the dosing regimen may involve alternating administration of Compound A and therapeutic agent over a period of several days, weeks, or months.

The combination therapy may provide “synergy” and “synergistic effect”, i.e., the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e., serially.

In some embodiments, the therapeutic agent is selected from the following group consisting of Bortezomib (VELCADE®), Carfilzomib (PR-171), PR-047, disulfuram, lactacystin, PS-519, eponemycin, epoxomycin, aclacinomycin, CEP-1612, MG-132, CVT-63417, PS-341, vinyl sulfone tripeptide inhibitors, ritonavir, PI-083, (+/−)-7-methylomuralide, (−)-7-methylomuralide, Perifosine, Rituximab, Sildenafil citrate (VIAGRA®), CC-5103, Thalidomide, Epratuzumab (hLL2-anti-CD22 humanized antibody), Simvastatin, Enzastaurin, Campath-1H, Dexamethasone, DT PACE, oblimersen, antineoplaston A10, antineoplason AS2-1, alemtuzumab, beta alethine, cyclophosphamide, doxorubicin hydrochloride, PEGylated liposomal doxorubicin hydrochloride, prednisone, prednisolone, cladribine, vincristine sulfate, fludarabine, filgrastim, melphalan, recombinant interferon alfa, carmustine, cisplatin, cyclophosphamide, cytarabine, etoposide, melphalan, dolastatin 10, indium In 111 monoclonal antibody MN-14, yttrium Y 90 humanized epratuzumab, anti-thymocyte globulin, busulfan, cyclosporine, methotrexate, mycophenolate mofetil, therapeutic allogeneic lymphocytes, Yttrium Y 90 ibritumomab tiuxetan, sirolimus, tacrolimus, carboplatin, thiotepa, paclitaxel, aldesleukin, recombinant interferon alfa, docetaxel, ifosfamide, mesna, recombinant interleukin-12, recombinant interleukin-11, Bcl-2 family protein inhibitor ABT-263, denileukin diftitox, tanespimycin, everolimus, pegfilgrastim, vorinostat, alvocidib, recombinant flt3 ligand, recombinant human thrombopoietin, lymphokine-activated killer cells, amifostine trihydrate, aminocamptothecin, irinotecan hydrochloride, caspofungin acetate, clofarabine, epoetin alfa, nelarabine, pentostatin, sargramostim, vinorelbine ditartrate, WT-1 analog peptide vaccine, WT1 126-134 peptide vaccine, fenretinide, ixabepilone, oxaliplatin, monoclonal antibody CD19, monoclonal antibody CD20, omega-3 fatty acids, mitoxantrone hydrochloride, octreotide acetate, tositumomab and iodine I 131 tositumomab, motexafin gadolinium, arsenic trioxide, tipifarnib, autologous human tumor-derived HSPPC-96, veltuzumab, bryostatin 1, anti-CD20 monoclonal antibodies, chlorambucil, metformin, rosiglitazone, pioglitazone, pentostatin, lumiliximab, apolizumab, Anti-CD40, Ofatumumab, bendamustine, and a combination thereof.

In other embodiments, the therapeutic agent is a proteasome inhibitor.

In a particular embodiment, the therapeutic procedure is selected from the group consisting of peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, immunohistochemistry staining method, pharmacological study, low-LET cobalt-60 gamma ray therapy, bleomycin, conventional surgery, radiation therapy, high-dose chemotherapy and nonmyeloablative allogeneic hematopoietic stem cell transplantation.

In a particular embodiment, the method further comprises obtaining a biological sample from the subject; and analyzing the biological sample with an analytical procedure selected from the group consisting of blood chemistry analysis, chromosomal translocation analysis, needle biopsy, fluorescence in situ hybridization, laboratory biomarker analysis, immunohistochemistry staining method, flow cytometry, genetic analysis, or a combination thereof. Analysis may provide information about which patients may benefit from therapy, regression or progression of the tumor, an appropriate duration of the treatment, and is useful for determining dosages to administer, for adjusting dosages during a treatment cycle, and for deciding whether to continue or discontinue the treatments. The subject may be any mammal, including human and non-human such as dogs. In some embodiments, the subject is a healthy person. In other embodiment, the subject is a patient having cancer or a condition related to PI3K-mediated disorders.

In one embodiment, the method described herein comprises administering to a subject Compound A described herein, in combination with a therapy used to treat cancer or a condition related to PI3K-mediated disorders. The “therapy” used to treat cancer or a condition related to PI3K-mediated disorders, as used herein, is any well-known or experimental form of treatment used to treat cancer or a condition related to PI3K-mediated disorders that does not include the use of Compound A. In certain embodiments, the combination of Compound A with a conventional or experimental therapy used to treat cancer or a condition related to PI3K-mediated disorders provides beneficial and/or desirable treatment results superior to results obtained by treatment without the combination. In certain embodiments, the therapies used to treat cancer or a condition related to PI3K-mediated disorders are well-known to a person having ordinary skill in the art and are described in the literature. Therapies include, but are not limited to, chemotherapy, combinations of chemoimmunotherapy, biological therapies, hormonal therapies, immunotherapy, radioimmunotherapy, monoclonal antibodies, and vaccines. In certain embodiments, the combination method provides for Compound A administered simultaneously or during the period of administration of the therapy. In certain embodiments, the combination method provides for Compound A administered prior to or after the administration of the therapy. The exact details regarding the administration of the combination may be determined experimentally. The refinement of sequence and timing of administering Compound A with a selected therapy will be tailored to the individual subject, the nature of the condition to be treated in the subject, and generally, the judgment of the attending practitioner.

Additional therapeutic agents for combinations with Compound A include those routinely used in the treatment of solid tumors, particularly Docetaxel, Mitoxantrone, Prednisone, Estramustine, Anthracyclines, (doxorubicin (Adriamycin), epirubicin (Ellence), and liposomal doxorubicin (Doxil)), Taxanes (docetaxel (Taxotere), paclitaxel (Taxol), and protein-bound paclitaxel (Abraxane)), Cyclophosphamide (Cytoxan), Capecitabine (Xeloda) and 5 fluorouracil (5 FU), Gemcitabine (Gemzar), methotrexate, Vinorelbine (Navelbine), an EGFR inhibitor such as erlotinib, Trastuzumab (Herceptin, this drug is only of use in women whose breast cancers have the HER-2 gene), Avastin, Platins (cisplatin, carboplatin), Temazolamide, Interferon alpha, and IL-2.

In certain embodiments, the method comprises administering to the subject, in addition to an effective amount of Compound A, at least one therapeutic agent and/or therapeutic procedure selected to treat the cancer or a condition related to PI3K-mediated disorders in the subject. In certain embodiments, the method comprises administering in addition to Compound A to the subject, a therapeutically effective amount of an additional therapeutic agent selected from Docetaxel, Mitoxantrone, Prednisone, Estramustine, Anthracyclines, (doxorubicin (Adriamycin), epirubicin (Ellence), and liposomal doxorubicin (Doxil)), Taxanes (docetaxel (Taxotere), paclitaxel (Taxol), and protein-bound paclitaxel (Abraxane)), Cyclophosphamide (Cytoxan), Capecitabine (Xeloda) and 5 fluorouracil (5 FU), Gemcitabine (Gemzar), methotrexate, Vinorelbine (Navelbine), an EGFR inhibitor such as erlotinib, Trastuzumab (Herceptin, this drug is only of use in women whose breast cancers have the HER-2 gene), Avastin, Platins (cisplatin, carboplatin), Temazolamide, Interferon alpha, and IL-2.

The compounds described herein may be formulated for administration to animal subjects using commonly understood formulation techniques well known in the art. Formulations which are suitable for particular modes of administration and for Compound A may be found in Remington's Pharmaceutical Sciences, latest edition, Mack Publishing Company, Easton, Pa.

The compounds described herein may be prepared in the form of prodrugs, i.e., protected forms which release the compounds described herein after administration to the subject. Typically, the protecting groups are hydrolyzed in body fluids such as in the bloodstream thus releasing the active compound or are oxidized or reduced in vivo to release the active compound. A discussion of prodrugs is found in Smith and Williams Introduction to the Principles of Drug Design, Smith, H. J.; Wright, 2^{nd} ed., London (1988).

A compound described herein can be administered as the neat chemical, but it is typically preferable to administer the compound in the form of a pharmaceutical composition or formulation. Accordingly, also provided are pharmaceutical compositions that comprise Compound A and a biocompatible pharmaceutical carrier, adjuvant, or vehicle. The composition can include the agent as the only active moiety or in combination with other agents, such as oligo- or polynucleotides, oligo- or polypeptides, drugs, or hormones mixed with excipient(s) or other pharmaceutically acceptable carriers. Carriers and other ingredients can be deemed pharmaceutically acceptable insofar as they are compatible with other ingredients of the formulation and not deleterious to the recipient thereof.

The pharmaceutical compositions are formulated to contain suitable pharmaceutically acceptable carriers, and can optionally comprise excipients and auxiliaries that facilitate processing of the active compounds into preparations that can be used pharmaceutically. The administration modality will generally determine the nature of the carrier. For example, formulations for parenteral administration can comprise aqueous solutions of the active compounds in water-soluble form. Carriers suitable for parenteral administration can be selected from among saline, buffered saline, dextrose, water, and other physiologically compatible solutions. Preferred carriers for parenteral administration are physiologically compatible buffers such as Hank's solution, Ringer's solution, or physiologically buffered saline. For tissue or cellular administration, penetrants appropriate to the particular barrier to be permeated are used in the formulation. Such penetrants are generally known in the art. For preparations comprising proteins, the formulation can include stabilizing materials, such as polyols (e.g., sucrose) and/or surfactants (e.g., nonionic surfactants), and the like.

Alternatively, formulations for parenteral use can comprise dispersions or suspensions of the active compounds prepared as appropriate oily injection suspensions. Suitable lipophilic solvents or vehicles include fatty oils, such as sesame oil, and synthetic fatty acid esters, such as ethyl oleate or triglycerides, or liposomes. Aqueous injection suspensions can contain substances that increase the viscosity of the suspension, such as sodium carboxy-methylcellulose, sorbitol, or dextran. Optionally, the suspension also can contain suitable stabilizers or agents that increase the solubility of the compounds to allow for the preparation of highly concentrated solutions. Aqueous polymers that provide pH-sensitive solubilization and/or sustained release of the active agent also can be used as coatings or matrix structures, e.g., methacrylic polymers, such as the EUDRAGIT™ series available from Rohm America Inc. (Piscataway, N.J.). Emulsions, e.g., oil-in-water and water-in-oil dispersions, also can be used, optionally stabilized by an emulsifying agent or dispersant (surface active materials; surfactants). Suspensions can contain suspending agents such as ethoxylated isostearyl alcohols, polyoxyethlyene sorbitol and sorbitan esters, microcrystalline cellulose, aluminum metahydroxide, bentonite, agar-agar, gum tragacanth, and mixtures thereof.

Liposomes containing the active agent also can be employed for parenteral administration. Liposomes generally are derived from phospholipids or other lipid substances. The compositions in liposome form also can contain other ingredients, such as stabilizers, preservatives, excipients, and the like. Preferred lipids include phospholipids and phosphatidyl cholines (lecithins), both natural and synthetic. Methods of forming liposomes are known in the art. See, e.g., Prescott (Ed.), METHODS IN CELL BIOLOGY, Vol. XIV, p. 33, Academic Press, New York (1976).

The pharmaceutical compositions comprising the agent in dosages suitable for oral administration can be formulated using pharmaceutically acceptable carriers well known in the art. The preparations formulated for oral administration can be in the form of tablets, pills, capsules, cachets, dragees, lozenges, liquids, gels, syrups, slurries, elixirs, suspensions, or powders. To illustrate, pharmaceutical preparations for oral use can be obtained by combining the active compounds with a solid excipient, optionally grinding the resulting mixture, and processing the mixture of granules, after adding suitable auxiliaries if desired, to obtain tablets or dragée cores. Oral formulations can employ liquid carriers similar in type to those described for parenteral use, e.g., buffered aqueous solutions, suspensions, and the like.

Preferred oral formulations include tablets, dragees, and gelatin capsules. These preparations can contain one or excipients, which include, without limitation:
a) diluents, such as sugars, including lactose, dextrose, sucrose, mannitol, or sorbitol;
b) binders, such as magnesium aluminum silicate, starch from corn, wheat, rice, potato, etc.;
c) cellulose materials, such as methylcellulose, hydroxypropylmethyl cellulose, and sodium carboxymethylcellulose, polyvinylpyrrolidone, gums, such as gum arabic and gum tragacanth, and proteins, such as gelatin and collagen;
d) disintegrating or solubilizing agents such as cross-linked polyvinyl pyrrolidone, starches, agar, alginic acid or a salt thereof, such as sodium alginate, or effervescent compositions;
e) lubricants, such as silica, talc, stearic acid or its magnesium or calcium salt, and polyethylene glycol;
f) flavorants and sweeteners;
g) colorants or pigments, e.g., to identify the product or to characterize the quantity (dosage) of active compound; and
h) other ingredients, such as preservatives, stabilizers, swelling agents, emulsifying agents, solution promoters, salts for regulating osmotic pressure, and buffers.

In some preferred oral formulations, the pharmaceutical composition comprises at least one of the materials from group (a) above, or at least one material from group (b) above, or at least one material from group (c) above, or at least one material from group (d) above, or at least one material from group (e) above. Preferably, the composition comprises at least one material from each of two groups selected from groups (a)-(e) above.

Gelatin capsules include push-fit capsules made of gelatin, as well as soft, sealed capsules made of gelatin and a coating such as glycerol or sorbitol. Push-fit capsules can contain the active ingredient(s) mixed with fillers, binders, lubricants, and/or stabilizers, etc. In soft capsules, the active compounds can be dissolved or suspended in suitable fluids, such as fatty oils, liquid paraffin, or liquid polyethylene glycol with or without stabilizers.

Dragée cores can be provided with suitable coatings such as concentrated sugar solutions, which also can contain gum arabic, talc, polyvinyl pyrrolidone, carbopol gel, polyethylene glycol, and/or titanium dioxide, lacquer solutions, and suitable organic solvents or solvent mixtures.

The pharmaceutical composition can be provided as a salt of the active agent. Salts tend to be more soluble in aqueous or other protic solvents than the corresponding free acid or base forms. Pharmaceutically acceptable salts are well known in the art. Compounds that contain acidic moieties can form pharmaceutically acceptable salts with suitable cations. Suitable pharmaceutically acceptable cations include, for example, alkali metal (e.g., sodium or potassium) and alkaline earth (e.g., calcium or magnesium) cations.

Compound A may form pharmaceutically acceptable acid addition salts with suitable acids. For example, Berge, et al., describe pharmaceutically acceptable salts in detail in J. Pharm. Sci., 66:1 (1977). The salts can be prepared in situ during the final isolation and purification of the compounds described herein or separately by reacting Compound A with a suitable acid.

Representative acid addition salts include, but are not limited to, acetate, adipate, alginate, citrate, aspartate, benzoate, benzenesulfonate, bisulfate, butyrate, camphorate, camphorolsulfonate, digluconate, glycerophosphate, hemisulfate, heptanoate, hexanoate, fumarate, hydrochloride, hydrobromide, hydroiodide, 2-hydroxyethanesulfonate (isothionate), lactate, maleate, methanesulfonate or sulfate, nicotinate, 2-naphthalenesulfonate, oxalate, pamoate, pectinate, persulfate, 3-phenylpropionate, picrate, pivalate, propionate, succinate, tartrate, thiocyanate, phosphate or hydrogen phosphate, glutamate, bicarbonate, p-toluenesulfonate, and undecanoate. Examples of acids that can be employed to form pharmaceutically acceptable acid addition salts include, without limitation, such inorganic acids as hydrochloric acid, hydrobromic acid, sulfuric acid, and phosphoric acid, and such organic acids as oxalic acid, maleic acid, succinic acid, and citric acid.

Compositions comprising a compound described herein formulated in a pharmaceutical acceptable carrier can be prepared, placed in an appropriate container, and labeled for treatment of an indicated condition. Accordingly, there also is contemplated an article of manufacture, such as a container comprising a dosage form of a compound described herein and a label containing instructions for use of the compound. Kits are also contemplated for the compounds and methods described herein. For example, the kit can comprise a dosage form of a pharmaceutical composition and a package insert containing instructions for use of the composition in treatment of a medical condition. In certain embodiments, the kit comprises Compound A and at least one therapeutic agent disclosed herein. In certain embodiments, the kit may further comprise at least one pharmaceutically acceptable excipient. In either case, conditions indicated on the label can include treatment of cancer.

Pharmaceutical compositions comprising Compound A can be administered to the subject by any conventional method, including parenteral and enteral techniques. Parenteral administration modalities include those in which the composition is administered by a route other than through the gastrointestinal tract, for example, intravenous, intraarterial, intraperitoneal, intramedullarly, intramuscular, intraarticular, intrathecal, and intraventricular injections. Enteral administration modalities include, for example, oral (including buccal and sublingual) and rectal administration. Transepithelial administration modalities include, for example, transmucosal administration and transdermal administration. Transmucosal administration includes, for example, enteral administration as well as nasal, inhalation, and deep lung administration; vaginal administration; and rectal administration. Transdermal administration includes passive or active transdermal or transcutaneous modalities, including, for example, patches and iontophoresis devices, as well as topical application of pastes, salves, or ointments. Parenteral administration also can be accomplished using a high-pressure technique, e.g., POWDERJECT™.

Surgical techniques include implantation of depot (reservoir) compositions, osmotic pumps, and the like. A preferred route of administration for treatment of inflammation can be local or topical delivery for localized disorders such as arthritis, or systemic delivery for distributed disorders, e.g., intravenous delivery for reperfusion injury or for systemic conditions such as septicemia. For other diseases, including those involving the respiratory tract, e.g., chronic obstructive pulmonary disease, asthma, and emphysema, administration can be accomplished by inhalation or deep lung administration of sprays, aerosols, powders, and the like.

Compound A can be administered before, during, or after administration of chemotherapy, radiotherapy, and/or surgery. The formulation and route of administration chosen will be tailored to the individual subject, the nature of the condition to be treated in the subject, and generally, the judgment of the attending practitioner.

The therapeutic index of Compound A can be enhanced by modifying or derivatizing the compounds for targeted delivery to cancer cells expressing a marker that identifies the cells as such. For example, the compounds can be linked to an antibody that recognizes a marker that is selective or specific for cancer cells, so that the compounds are brought into the vicinity of the cells to exert their effects locally, as previously described (see for example, Pietersz, et al., Immunol. Rev., 129:57 (1992); Trail et al., Science, 261:212 (1993); and Rowlinson-Busza, et al., Curr. Opin. Oncol., 4:1142 (1992)). Tumor-directed delivery of these compounds enhances the therapeutic benefit by, inter alia, minimizing potential nonspecific toxicities that can result from radiation treatment or chemotherapy. In another aspect, Compound A and radioisotopes or chemotherapeutic agents can be conjugated to the same anti-tumor antibody.

The characteristics of the agent itself and the formulation of the agent can influence the physical state, stability, rate of in vivo release, and rate of in vivo clearance of the administered agent. Such pharmacokinetic and pharmacodynamic information can be collected through preclinical in vitro and in vivo studies, later confirmed in humans during the course of clinical trials. Thus, for any compound used in the method described herein, a therapeutically effective dose can be estimated initially from biochemical and/or cell-based assays.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the “therapeutic index,” which typically is expressed as the ratio LD50/ED50. Compounds that exhibit large therapeutic indices (i.e., the toxic dose is substantially higher than the effective dose) are preferred. The data obtained from such cell culture assays and additional animal studies can be used in formulating a range of dosage for human use. The dosage of such compounds lies preferably within a range of circulating concentrations that include the ED₅₀ with little or no toxicity.

For the methods described herein, any effective administration regimen regulating the timing and sequence of doses can be used. Doses of the agent preferably include pharmaceutical dosage units comprising an effective amount of the agent. As used herein, “effective amount” refers to an amount sufficient to modulate the PI3Kα-sparing activity or any combination of PI3Kδ, PI3Kγ, and PI3Kβ expression or activity and/or derive a measurable change in a physiological parameter of the subject through administration of one or more of the pharmaceutical dosage units. “Effective amount” can also refer to the amount required to ameliorate a disease or disorder in a subject.

Suitable dosage ranges for Compound A may vary according to these considerations, but in general, Compound A may be administered in the range of about 10.0 μg/kg to 15 mg/kg of body weight; about 1.0 μg/kg to 10 mg/kg of body weight, or about 0.5 mg/kg to 5 mg/kg of body weight. For a typical about 70-kg human subject, thus, the dosage range is from about 700 μg to 1050 mg; about 70 μg to 700 mg; or about 35 mg to 350 mg per dose, and two or more doses may be administered per day. Dosages may be higher when Compound A is administered orally or transdermally as compared to, for example, i.v. administration. In certain embodiments, the treatment of cancers comprises oral administration of up to about 750 mg/day of Compound A. The reduced toxicity of this compound permits the therapeutic administration of relatively high doses. The reduced toxicity of Compound A, permits the therapeutic administration of relatively high doses. For treatment of leukemias and lymphomas and multiple myeloma, a dosage of about 50 to 100 mg per dose, administered orally once or preferably twice per day, is often suitable. For treatment of many solid tumors, a dosage of about 50 to 100 mg per dose, administered orally once or preferably at least twice per day, is often suitable. In some embodiments, Compound A is administered orally, in three to five doses per day, using about 20 to 150 mg per dose for a total daily dose between about 60 to 750 mg. In some embodiments, the total daily dose is between about 100 to 500 mg, and in some embodiments the normalized daily dosage (adjusted for subject's body weight) is up to about 60 mg per kg of the treated subject's body weight.

Compound A may be administered as a single bolus dose, a dose over time, as in i.v. or transdermal administration, or in multiple dosages. For i.v. or transdermal delivery, a dosage may be delivered over a prolonged period of time, and may be selected or adjusted to produce a desired plasma level of the active compound. In some embodiments, the desired plasma level is at least about 1 micromolar, or at least about 10 micromolar.

When Compound A is administered orally, it is preferably administered one time per day or in two or more doses per day. In some embodiments, three doses per day are administered. In some embodiments four doses per day are administered.

Dosing may be continued for one day or for multiple days, such as about 7 days. In some embodiments, daily dosing is continued for about 14 days or about 28 days. In some embodiments, dosing is continued for about 28 days and is then discontinued for about 7 days; the efficacy of the treatment can be assessed during the break, when treatment with Compound A has been stopped, and if the assessment shows that the treatment is achieving a desired effect, another cycle of about 7 to 28 days of treatment with Compound A can be initiated.

Depending on the route of administration, a suitable dose can be calculated according to body weight, body surface area, or organ size. The final dosage regimen will be determined by the attending physician in view of good medical practice, considering various factors that modify the action of drugs, e.g., the agent's specific activity, the identity and severity of the disease state, the responsiveness of the patient, the age, condition, body weight, sex, and diet of the patient, and the severity of the cancer, a condition related to PI3K-mediated disorders, or any infection. Additional factors that can be taken into account include comorbidities, prior therapies, the time and frequency of administration, drug combinations, reaction sensitivities, and tolerance/response to therapy. Further refinement of the dosage appropriate for treatment involving any of the formulations mentioned herein is done routinely by the skilled practitioner without undue experimentation, especially in light of the dosage information and assays disclosed, as well as the pharmacokinetic data observed in human clinical trials. Appropriate dosages can be ascertained through use of established assays for determining concentration of the agent in a body fluid or other sample together with dose-response data.

The frequency of dosing will depend on the pharmacokinetic parameters of the agent and the route of administration. Dosage and administration are adjusted to provide sufficient levels of the active moiety or to maintain the desired effect. Accordingly, the pharmaceutical compositions can be administered in a single dose, multiple discrete doses, continuous infusion, sustained release depots, or combinations thereof, as required to maintain desired minimum level of the agent. Short-acting pharmaceutical compositions (i.e., short half-life) can be administered once a day or more than once a day (e.g., two, three, or four times a day). Long acting pharmaceutical compositions might be administered every 3 to 4 days, every week, or once every two weeks to 12 weeks. Pumps, such as subcutaneous, intraperitoneal, or subdural pumps, can be preferred for continuous infusion.

Subjects that will respond favorably to the methods described herein include medical and veterinary subjects generally, including human patients. Among other subjects for whom the methods described herein are useful are cats, dogs, large animals, avians such as chickens, and the like. In general, any subject who would benefit from Compound A is appropriate for administration of the method described herein.

The present disclosure will be understood more readily by reference to the following examples, which are provided by way of illustration and are not intended to be limiting of the present disclosure.

### EXAMPLES

### Example 1

### Preparation of Compound A

The (S)-enantiomer of Compound A was prepared as shown in Scheme 1.

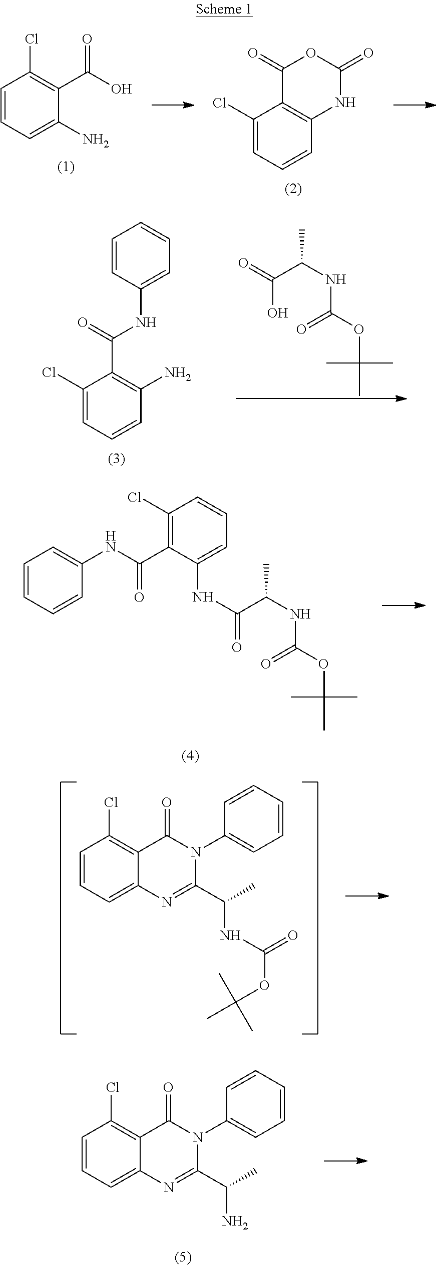

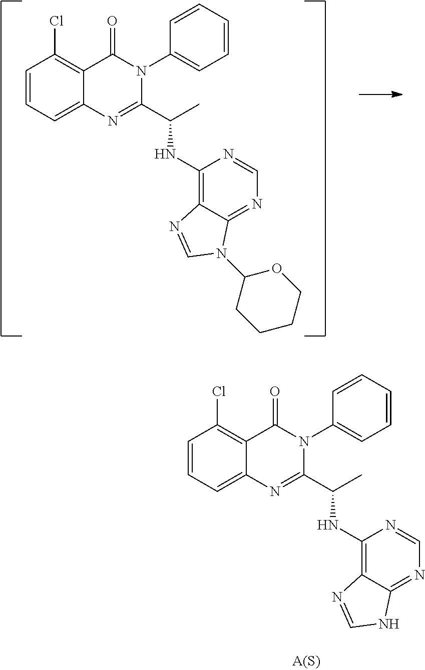

### Preparation of 5-chloro-2H-3,1-benzoxazine-2,4(1H)-dione (2)

2-amino-6-chlorobenzoic acid (1) (10 g, 1 equiv) was dissolved in acetonitrile (58.1 mL, 19.1 equiv) at 50° C., and added with pyridine (9.4 mL, 2 equiv). Then, triphosgene (5.7 g, 0.33 equiv) in methylene chloride (30 mL, 9 equiv) was added dropwise with stiffing. The reaction mixture was stirred for 2 hours at 50° C. The solvent was removed by rotary evaporation. The residue was then dispersed in 50 ml water and filtered. The tan solid was washed with a minimal amount of acetonitrile to remove color, and dried to produce an off-white solid powder. HPLC RT was 4.41 minutes. All compounds were characterized using high performance liquid chromatograph (HPLC), with elution from the Zorbax C₈ column using a gradient of 0-100% acetonitrile in water containing 0.07% trifluoroacetic acid (TFA) and detection using absorbance at 210 nm and 254 nm.

### Preparation of 2-amino-6-chloro-N-phenylbenzamide (3)

5-chloro-2H-3,1-benzoxazine-2,4(1H)-dione (2) (2.00 g, 1 equiv) was dissolved in dioxane (15 mL, 19 equiv) at 40° C. The aniline (1.38 mL, 1 equiv) was added dropwise over 30 minutes, gradually warming to 100° C. The reaction mixture was stirred for 4 hours then cooled to ambient temperature (25° C.±5° C.). The solvent was removed by evaporation. Chromatography was performed using 90 g silica gel with 1:1 (v/v) ethyl acetate:hexane to yield a white solid. HPLC RT was 5.46 minutes.

### Preparation of (S)-tert-butyl 1-(3-chloro-2-(phenylcarbamoyl)phenylamino)-1-oxopropan-2-ylcarbamate (4)

(S)-2-(tert-butoxycarbonylamino)propanoic acid (0.4 g, 2 equiv) was dissolved in dry tetrahydrofuran (THF)(3 mL, 40 equiv), and 4-methylmorpholine (0.256 mL, 2.2 equiv) was added. The reaction mixture was then cooled to −15° C. in an ethylene glycol/CO₂ bath. A solution of isobutyl chloroformate (0.274 mL, 2 equiv) in dry THF (1 mL) was added dropwise to the reaction mixture, and stirred for 30 minutes. The reaction was stirred at −15° C. for 30 minutes, then added with 2-amino-6-chloro-N-phenylbenzamide (3) in THF (1.0 mL). The reaction mixture was slowly warmed to 21° C. When about 10% conversion was observed, the reaction mixture was warmed to 60° C. for 30 minutes. The reaction mixture was poured into ethyl acetate (150 mL), and washed with water (50 mL) twice and brine (30 mL). The organic layer was dried over sodium sulfate, filtered, and rotary evaporated to remove the solvent. Chromatography was performed using 90 g silica gel with 1:4 (v/v) ethyl acetate:hexane to yield white crystals.

### Preparation of (S)-2-(1-aminoethyl)-5-chloro-3-phenylquinazolin-4(3H)-one (5)

(S)-tert-butyl-1-(3-chloro-2-(phenylcarbamoyl)phenylamino)-1-oxopropan-2-ylcarbamate (4) (5 g, 1 equiv) was dissolved in acetonitrile (300 mL, 500 equiv) under a nitrogen atmosphere. Triethylamine (79.21 mL, 47.5 equiv) was added with stiffing, followed by the dropwise addition of chlorotrimethylsilane (22.78 mL, 15 equiv). The flask was sealed, and placed in an oil bath and heated to 90° C. for 48 hours. HPLC RT was 6.66 minutes. The solvents were evaporated, and the dark residue was dissolved in ethyl acetate (350 mL) and washed with sodium bicarbonate (100 mL), water (100 mL) and brine (100 mL). The organic layer was dried over sodium sulfate, filtered and concentrated by rotary evaporation to yield a brown solid (tert-butyl [(1S)-1-(5-chloro-4-oxo-3-phenyl-3,4-dihydroquinazolin-2-yl)ethyl]carbamate).

The brown solid was then dissolved in methylene chloride (15 mL). TFA (6 mL) was added at 21° C. and stirred for 2 hours. The reaction mixture was diluted with toluene (100 mL), and solvents were removed by rotary evaporation. The remaining solid was dissolved in ethyl acetate (300 mL), and washed with sodium bicarbonate (100 mL), water (100 mL), and brine (100 mL). The organic layer was dried over magnesium sulfate, filtered, and rotary evaporated to remove solvent. Chromatography was performed using 40 g silica gel with chloroform and a slow gradient to 10% methanol (containing 10% ammonium hydroxide)-chloroform to yield (S)-2-(1-aminoethyl)-5-chloro-3-phenylquinazolin-4(3H)-one (5).

### Preparation of (S)-5-chloro-3-phenyl-2-[(1S)-1-(9H-purin-6-ylamino)ethyl]quinazolin-4(3H)-one-A(S)

N,N-diisopropylethylamine (2.74 mL, 3 equiv) was added to a stirred suspension of 2-[(1S)-1-aminoethyl]-5-chloro-3-phenylquinazolin-4(3H)-one (5) (1.574 g, 1 equiv) and 6-chloro-9-(tetrahydro-2H-pyran-2-yl)-9H-purine (1.38 g, 1.1 equiv) in isopropyl alcohol (10 mL, 30 equiv) contained in a small pressure tube under nitrogen. The tube was sealed, placed in an oil bath, and then heated to 80° C. over about 10-15 minutes. The programmed oil bath was turned off after 36 hours.

Water (100 mL) was added to the reaction slurry, and stirred for 15 minutes. The reaction mixture was then cooled in ice for 15 minutes, and then filtered and washed with water to yield about 6 g of a cream solid (wet). The solid was then dissolved in ethyl acetate (100 mL) and washed with brine (30 mL). The organic layer was dried over magnesium sulfate, filtered, and evaporated to yield 2.7 g of a yellow foam. This foam was dissolved in methylene chloride, and chromatography was performed using 120 g Biotage SiO₂ column with 200 mL methylene chloride, then 400 mL each of 25% ethyl acetate, 50% ethyl acetate, 75% ethyl acetate, 100% ethyl acetate, 500 mL 2% methanol/ethyl acetate, and 500 mL 4% methanol/ethyl acetate. White foam of purified materials (2.1 g) was collected, and dissolved in methylene chloride (50 mL) to which TFA (10 mL) was added. The colorless solution was observed to immediately form a pale yellow solution, and within 10-15 minutes, the solution was observed to have a rose wine color that slowly became darker. Toluene (250 mL) was added, and solvents were evaporated at 40° C. The residue was dissolved in ethyl acetate (250 mL), and washed with saturated sodium bicarbonate (75 mL) and brine (50 mL). The organic layer was dried over magnesium sulfate, filtered, and evaporated to yield 2.5 g of dirty yellow solid.

The yellow solid was dissolved in methylene chloride and methanol, and injected on to dry 90 g Biotage SiO₂ column. After blow drying for 45 minutes, the column was eluted with 500 mL methylene chloride, followed by 250 mL each of 2%, 4%, 6%, 8%, and 10% of methanol (containing 10% ammonium hydroxide)-chloroform. The column yielded 2 g of yellow foam, which was dissolved in methanol, filtered, and then evaporated. Crystals were observed in the filtrate, and the volume of the filtrate was reduced to 1-2 mL. The reduced filtrate was then allowed to sit for 5 min. Ether (20-25 mL) was slowly added. The solid was scraped from flask, washed with ether, and dried to yield 1.31 g of Compound A(S). HPLC RT was 4.67 minutes. Compound A(S) was characterized as shown in Table 1. ¹H-NMR (CDCL3): 8.362 (s, 1H), 8.028 (s, 1H), 7.662-7.532 (m, 6H), 7.493-7.471 (m, 1H), 7.395-7.376 (m, 1H), 7.073 (br s, 1H), 5.233 (br s, 1H), 1.542-1.525 (d, 3H).

TABLE 1
Summary of Compound A(S) characteristics

| Test | Test Result |
|---|---|
| Appearance | White powder to cream color |
| | powder |
| 1H-NMR | Consistent with structure |
| HPLC Assay | 97% at 210 nm |
| | 100% at 254 nm |
| Mass Spectrum | Consistent with structure |
| | M + H = 418.2 |
| | M − H = 416.2 |

In the examples below, unless stated otherwise, Compound A is the optically active form that predominantly includes the (S)-enantiomer.

### Example 2

### In Vivo Pharmacokinetics of Compound A in Rats, Mice and Dogs

Compound A was administered as a single dose at 1 mg/kg for intravenous dosing (IV), and at 3 and 30 mg/kg for oral dosing (PO) in rats as shown in Table 2.

TABLE 2
Conditions for in vivo PK study

| | | Dosing | Dosing | Sample time |
|---|---|---|---|---|
| Assay | Dose | volume | technique | points |
| In vivo PK | 1 mg/kg (n = 3) | 5 mL/kg | Jugular | 0.08, 0.25, 0.5, |
| (cannulated | | | catheter | 1, 2, 4, 6 |
| rat, IV) | | | | and 24 hour |
| In vivo PK | 3 and 30 | 5 mL/kg | Gastric | 0.25, 0.5, 1, 2, |
| (cannulated | mg/kg (n = 3) | | gavage | 4, 6, 8 |
| rat, PO) | | | | and 24 hour |

Over a period of 24 hours, the plasma levels of Compound A peaked at about 2-4 hours after administration (data not shown). The pharmacokinetic parameters were shown in Table 3.

TABLE 3
Pharmacokinetic parameters in rats administered with Compound A.

| Route of | Dose | | T½ | CL | Vz | Vss | AUClast | AUCINF | Terminal |
|---|---|---|---|---|---|---|---|---|---|
| Administration | (mg/kg) | Subject | (min) | (mL/min/kg) | (mL/kg) | (mL/kg) | (min * ng/mL) | (min * ng/mL) | Points |
| IV | 1 | Rat 7 | 4.3 | 951 | 5849 | 2638 | 1043 | 1052 | 3 |
| | | Rat 8 | 1.4 | 689 | 1362 | 1320 | 1389 | 1430 | 6 |
| | | Rat 9 | 1.1 | 1089 | 1768 | 1674 | 896 | 918 | 5 |
| | | Mean | 2.2 | 913 | 2983 | 1877 | 1103 | 1133 | |
| | | SE | 1.0 | 114 | 1433 | 394 | 140 | 153 | |

| Route of | Dose | | Bioavailability | Tmax | Cmax | T½ | AUClast | AUCINF | Terminal |
|---|---|---|---|---|---|---|---|---|---|
| Administration | (mg/kg) | Subject | (%) | (min) | (ng/mL) | (min) | (min * ng/mL) | (min * ng/mL) | Points |
| PO | 3 | Rat 1 | 81 | 1.0 | 568 | 2.9 | 2736 | 2749 | 3 |
| | | Rat 2 | 59 | 2.0 | 457 | 3.3 | 1993 | 2004 | 3 |
| | | Rat 3 | 37 | 1.0 | 345 | 4.5 | 1255 | 1273 | 3 |
| | | Mean | 59 | 1.3 | 456 | 3.5 | 1995 | 2009 | |
| | | SE | 13 | 0.3 | 64 | 0.5 | 426 | 426 | |
| | 30 | Rat 4 | 138 | 4.0 | 5798 | 2.27 | 48722 | 46792 | 3 |
| | | Rat 5 | 128 | 4.0 | 5770 | 2.27 | 43663 | 43729 | 3 |
| | | Rat 6 | 87 | 2.0 | 4320 | 2.30 | 29569 | 29610 | 3 |
| | | Mean | 118 | 3.3 | 5295 | 2.28 | 39985 | 40044 | |
| | | SE | 16 | 0.7 | 498 | 0.01 | 5292 | 5291 | |

In rats administrated intravenously (IV) with a dose of 1 mg/kg, the mean of elimination half-life (T1/2) was 2.2 min, the mean of total body clearance (CL) was 913 mL/min/kg, and mean of volume of distribution (Vz) was 2,993 mL/kg. Additionally, in rats administered orally (PO) with a dose of 3 mg/kg, the mean T1/2 was 3.6 min, the mean time of maximum observed concentration (Tmax) was 1.3 min, and mean bioavailability was 59%. See Table 4. Also, in rats administered orally (PO) with a dose of 30 mg/kg, the mean T1/2 was 2.28 min, mean Tmax was 3.3 min, and mean bioavailability was 118%.

The pharmacokinetics of Compound A in mouse and dogs were also examined. Compound A was dosed at 1 mg/kg for intravenous (IV) dosing, and at 1, 3, and 20 mg/kg for oral (PO) dosing. The results of Tmax (time of maximum observed concentration), Cmax (maximum concentration in plasma measured), AUC (area under the curve for plasma concentration versus time), Cl (total body clearance), and Vz (volume of distribution) were summarized in Table 4. As used herein, the mark ‘-’ represents data not relevant. AUC is shown as the unit of ng*h/mL (ng=nanograms, h=hour, mL=milliliter); ng are multiplied by h, and the value is divided by the volume in mL.

TABLE 4
ADME and pharmacokinetics data in mice, rats, and dogs.

| | | | | AUC | | |
|---|---|---|---|---|---|---|
| | | Tmax | Cmax | [ng * h/ | CL | V_{z} |
| | | [h] | [ng/mL] | mL] | [mg/h] | [mL/kg] |
| In vitro | Stable in human liver microsomes | | | | | |
| metabolism | | | | | | |
| Mouse PK | PO 20 mg/kg | 0.25 | 1284 | 1500 | — | — |
| Rat PK | IV 1 mg/kg | — | — | — | 913 | 2993 |
| Rat PK | PO 3 mg/kg | 1.3 | 456 | 1995 | — | — |
| Dog PK | IV 1 mg/kg | — | — | — | 365 | 891 |
| Dog PK | PO 1 mg/kg | 2.3 | 1040 | 4450 | — | — |

### Materials and Methods for Examples 3-10

Cell Lines, Antibodies, and Plasmid Constructs.

CCRF-CEM cells were obtained from ATCC and grown in RPMI-1640 medium containing 10% FBS and antibiotics.

Antibodies to Akt (catalog #9272), phospho-Akt (S473, clone 193H12), phospho-mTOR (S2448, catalog #2971S), mTOR (catalog #2972), phospho-GSK3α/β (S21/9, catalog #93315), GSK-313 (clone 27C10), phospho-p70S6K (Thr389, catalog #9205S) and p70S6K (catalog #9202) and β-actin (catalog #4967S) were from Cell Signaling Technology. Antibodies to class I PI3K subunits were as follows: p110α (catalog #4255) from Cell Signaling Technology; p11013 (clone Y384) from Millipore and mouse p11013 from Santa Cruz Biotechnology (catalog #sc-602); p110γ (clone H1) from Jena Biosciences; p110δ (clone H-219) from Santa Cruz Biotechnology. Antibodies to PTEN (clone 6H2.1) were from Cascade Bioscience. For flow cytometry, antibodies were obtained from BD Biosciences: CD3ε-Alexa 488 (clone 145-2C11), CD4-APC (clone RM4-5), CD8-PerCP-Cy5.5 (clone 53-6.7), CD90.2-APC (Thy-1.2, clone 53-2.1), Ki67-FITC (clone B56), and Annexin V-APC. Antibodies to Bim, phospho-Bad, Bad, and BclX_{L} were from Cell Signaling Technology (pro-apoptotic sampler kit #9942S).

The shRNA construct for p110γ in the pLKO.1 vector was obtained from Sigma (MISSION® shRNA Plasmid DNA; clone ID: NM_{—}002649.2-4744s1c1; TRC number: TRCN0000196870).

Primary Leukemia Samples.

Cryopreserved samples from the Columbia Presbyterian Hospital, the Erasmus MC-Sophia Children's Hospital, and the University of Padua were used. All samples were collected with informed consent and supervised by the institutes' review boards and the Acute Lymphoblastic Leukemia Strategic Scientific Committee.

Cell Counts and Proliferation Assays.

Cell counts for mice thymii were determined as described in Swat, W. et al., Essential role of PI3 Kdelta and PI3 Kgamma in thymocyte survival, Blood 107, 2415-2422 (2006). Cell proliferation of untransfected or shRNA transfected CCRF-CEM cells was followed by cell counting of samples in triplicate using a hemocytometer.

Cell Viability Assays.

For primary T-ALL samples, cell viability was determined using the BD Cell Viability kit (BD Biosciences) and fluorescent counting beads as previously described in Armstrong, F. et al., NOTCH is a key regulator of human T-cell acute leukemia initiating cell activity, Blood 113, 1730-1740 (2009). Cells were plated with MS5-DL1 stroma cells. After 72 hours following treatment, cells were harvested and stained with an APC-conjugated anti-human CD45 according to the manufacturer's instructions. For solid tumor cell lines, cellular viability was determined using the AlamarBlue kit (Invitrogen). About 10,000 cells in 100 μL of media containing 10% FBS were aliquoted into individual wells of a 96-well plate and treated with vehicle (DMSO) or compound in triplicate for 24 hours. Ten μL of AlamarBlue reagent was added to each well and incubated for 4 hours at 37° C. with 5% CO₂. Fluorescence was measured with an excitation wavelength at 530 to 560 nm and emission wavelength at 590 nm using Spectramax M5 plate reader (Molecular Devices, Sunnyvale, Calif.).

Apoptosis Analysis.

Cells were stained with APC-conjugated Annexin-V (BD Biosciences) in Annexin Binding Buffer (Miltyeni Biotec) and analyzed by flow cytometry.

EC₅₀.

To analyze PI3K p110α and p110β signaling, murine embryo fibroblast (MEFs) were removed from FBS and starved for 2 hours followed by stimulation with 10 ng/mL of PDGF (Cell Signaling Technologies, Danvers, Mass.) or 10 μM of LPA (Echelon, Salt Lake City, Utah) for 10 minutes at 37° C., respectively. After washing once in cold phosphate-buffered saline (PBS), the cell pellet was resuspended in 1× cell lysis buffer (Cell Signaling Technologies) supplemented with mini protease inhibitor mix (Roche, Indianapolis, Ind.), phosphate inhibitor cocktail set I and II (Calbiochem, San Diego, Calif.) for 15 minutes on ice. Whole-cell lysates were obtained by centrifugation at 16,000 g for 10 minutes at 4° C., and the soluble protein was analyzed by Western blotting for Akt and pAkt levels. To analyze PI3K p110δ and p110γ signaling, basophil activation was measured in isolated PBMC or whole blood using the Flow2 CAST kit according to the manufacture's standardized methods (Buhlman Laboratories AG, Switzerland). Briefly, p110δ was activated with anti-FCεRI and p110γ was activated with fMLP (2 μM) in the absence or presence of compounds. To monitor the basophil cell population and cellular activation, anti-CD63-FITC and anti-CCR3-PE antibodies were added to each sample. Cells were fixed and analyzed on a FC500 MPL flow cytometer (Beckman Coulter, Brea, Calif.).

IC₅₀.

IC₅₀ values for inhibiting PI3K isoforms were determined using in vitro SelectScreen kinase inhibitor assay service (Invitrogen Ltd.). Compounds were diluted in 10 mM of DMSO, and measured for 10-point kinase inhibitory activities over a range of concentration from 5 to 10⁴ nM with ATP concentration consistent with each enzyme's Kₘ.

Calcium Flux Measurements in Thymocytes.

Ca²⁺ flux measurements in single cell suspensions of mouse thymocytes were performed as described in Swat, W. et al., Essential role of PI3 Kdelta and PI3 Kgamma in thymocyte survival, Blood 107, 2415-2422 (2006) Inhibition of Ca²⁺ flux was measured after 30 minutes incubation with compounds at room temperature.

Flow Cytometry for Cell Surface Staining and Apoptosis.

Mouse whole blood was incubated with appropriate antibodies and processed using the BD Bioscience BD FACS Lysing Solution according to the manufacturer's instructions. Immediately after lysis, cells were permeabilized with 0.025% Tween-20 in lysing solution for 15 minutes, then incubated with Ki67 antibodies. Single cell suspensions of thymocytes were isolated and stained with the appropriate antibodies as described in Swat, W. et al., Essential role of PI3 Kdelta and PI3 Kgamma in thymocyte survival, Blood 107, 2415-2422 (2006).

Histological and immunohistochemical study. Formalin-fixed paraffin-embedded 5 μm tissue sections were stained with hematoxylin and eosin for histological diagnosis. For immunohistochemistry, anti-Ki67 (rabbit monoclonal, Abcam) and anti-CD3 (rabbit polyclonal, Dako) staining on similar tissue sections were performed after antigen retrieval by microwave heating in citrate buffer (pH 6.0). After epitope recovery, slides were incubated with antibody (anti-Ki67 1:50, anti-CD3 1:50) overnight at room temperature before antigen detection with diaminobenzidine (DAB) using a Ventana automated staining platform (Ventana).

Immunoblot Analysis.

Cell lysates (from cell lines or thymocytes) were prepared on ice in M-PER Mammalian Protein Extraction reagent (Pierce) containing a cocktail of protease and phosphatase inhibitors as described in Swat, W. et al., Essential role of PI3 Kdelta and PI3 Kgamma in thymocyte survival, Blood 107, 2415-2422 (2006). Equal amounts of total protein from lysates were separated using SDS-PAGE, transferred to PVDF membrane (Immobilon-P, Millipore). Membranes were incubated overnight incubation with appropriate primary antibodies. Bound antibodies were visualized with HRP-conjugated secondary antibodies and ECL chemistry (SuperPico West, Pierce).

Animal Procedures.

All mice were kept in specific pathogen-free facility at Columbia University Medical Center. All procedures were performed in accordance with the guidelines of the Institutional Animal Care and Use Committee. Lck-cre, NOD.Cg-Prkdc^{scid} Il2rg^{tmlWjl}/Sz and Gt(ROSA)26Sor^{tml(Luc)Kael}/J mice were obtained from the Jackson Laboratory. Mice deficient for PTEN in the T-cell lineage were generated by crossing Lck-cre with floxed PTEN. P110γ^{-/-} and p110δ^{-/-} mice as described in Swat, W. et al., Essential role of PI3 Kdelta and PI3 Kgamma in thymocyte survival, Blood 107, 2415-2422 (2006). The animals were intercrossed with Lck-cre/PTEN^{fl/fl} animals to generate mice homozygous mutant for either p110γ or p110δ and PTEN or homozygous mutant for p110γ, 110δ, and PTEN.

Subcutaneous Xenograft Transplantation.

Luminescent CCRF-CEM (CEM-luc) cells were generated by lentiviral infection with FUW-luc and selection with neomycin. Luciferase expression was verified with the Dual-Luciferase Reporter Assay kit (Promega). CEM-luc cells (2.5×10⁶) embedded in Matrigel (BD Biosciences) were injected in the flank of NOD.Cg-Prkdc^{scid} Il2rg^{tmlWjl}/Sz mice. After 1 week, mice were treated by oral gavage with vehicle (0.5% methyl cellulose, 0.1% Tween-80) or compound every 8 hours daily for 4 days. Mice were anesthetized by isoflurane inhalation by intraperitoneal injection of D-luciferin (50 m/kg, Xenogen). Photonic emission was imaged with the In Vivo Imaging System (IVIS, Xenogen). Tumor bioluminescence was quantified by integrating the photonic flux (photons per second) through a region encircling each tumor using the LIVING IMAGES software package (Xenogen).

Intravenous Xenograft Transplantation.

CCRF-CEM cells (5×10⁶) were injected intravenously in fourteen NOD.Cg-Prkdc^{scid} Il2rg^{tmlWjl}/Sz mice. After 3 days, mice were segregated into two groups that received compound or vehicle for 7 days. Mice in both groups were then followed until moribund and euthanized.

Plasma Levels.

Plasma was collected at 0, 2, 4, 8, and 12 hours, and analyzed using HPLC/MS (sensitivity 1 ng/mL). The concentration of compound in plasma was determined using a standard curve (analyte peak area versus concentration) generated with calibration standard pools. Values represent the mean (±s.d.) for four animals per group.

shRNA Knock-Down of p110γ.

CCRF-CEM cells (2×10⁶) were transfected with purified plasmid DNA (2 μg) using the Amaxa® Human T-cell Nucleofector® Kit (Lonza) following the manufacturer's optimized protocol kit for CCRF-CEM cells. Clones were selected by high dilution in puromycin used at a concentration pre-determined by a killing curve. Expression of p110γ was determined by immunoblot analysis.

Statistical Analyses.

Statistical analyses were performed using Student's t-test (GraphPad Prizm software). Kaplan-Meier survival curves were analyzed using a log-rank test (GraphPad Prism software). Values were considered significant at P<0.5.

### Example 3

### Effect of PI3Kγ and PI3Kδ Activity on Malignant Transformation of T-Cells

This example shows that PI3Kγ and PI3Kδ support lymphomagenesis in the context of PTEN deficiency, and demonstrates the persistence of cellular and structural defects in thymi associated with a combined deletion of PI3K p110γ/δ and PTEN. PTEN (phosphatase and tensin homolog) is a non-redundant plasma-membrane phosphatase and responsible for counteracting potential cancer-promoting activities of class I PI3K by limiting the levels of PIP₃ which is induced by PI3K activation.

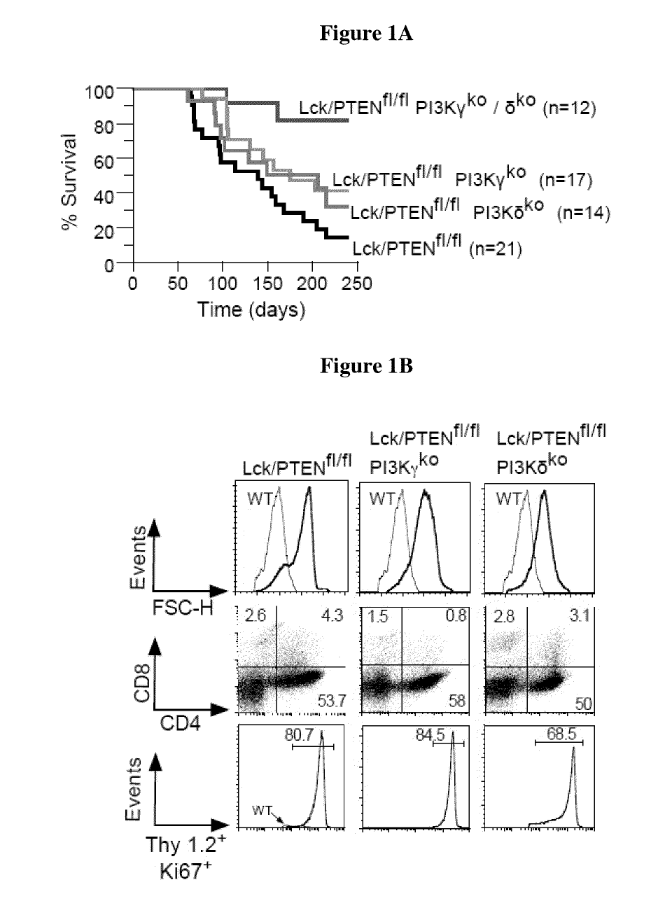

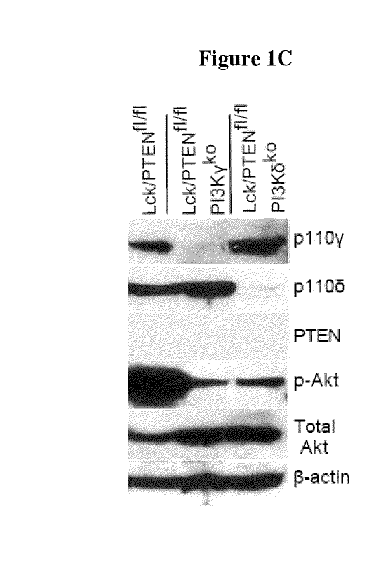

Mice having PTEN alleles floxed by the loxP Cre excision sites were crossed with the Lck-cre transgenic animals to generate Lck/PTEN^{fl/fl} mice; or cross with the Lck-cre transgenic animals lacking p110γ, p110δ, or both p110γ/p110δ to generate Lck/PTEN^{fl/fl} PI3Kγ^{ko}, Lck/PTEN^{fl/fl} PI3Kδ^{ko}, or Lck/PTEN^{fl/fl} PI3Kδ^{ko}γ^{ko} mice, respectively. More than 85% of Lck/PTEN^{fl/fl} mice developed T-cell acute lymphoblastic leukemia (T-ALL) and had the median survival of 140 days (FIG. 1A). The onset of disease and survival were improved in Lck/PTEN^{fl/fl} PI3Kδ^{ko}γ^{ko} mice that less than about 20% of animals developed T-ALL and had median survival of 220 days. The T-ALL development and medium survival was also increased in triple mutant mice: 65% and 175 days in Lck/PTEN^{fl/fl} PI3Kγ^{ko}; 64% and 178 days in Lck/PTEN^{fl/fl} PI3Kδ^{ko} mice. The results showed that either PI3Kγ and δ isomer was involved in tumorigenesis. While the activation in the triple mutants was lower as compared to those from Lck/PTEN^{fl/fl} animals, the PI3K/Akt signaling pathway was activated in all examined mice. The results showed that individual PI3K isomer mutant did not reduce proliferating blast.

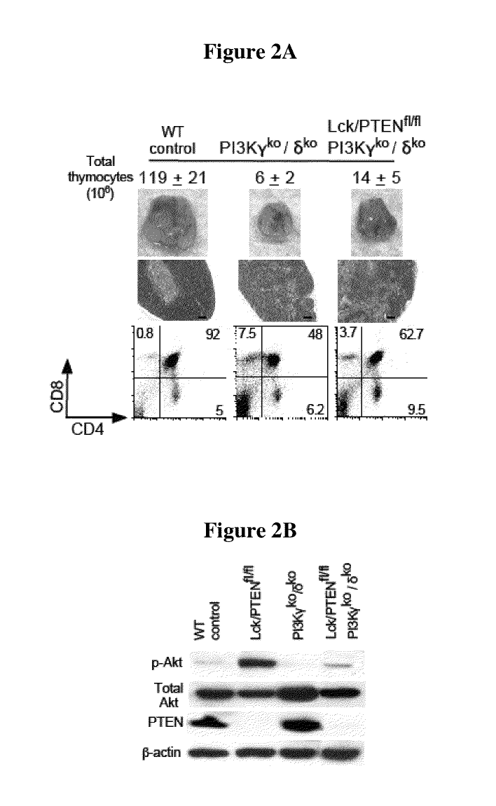

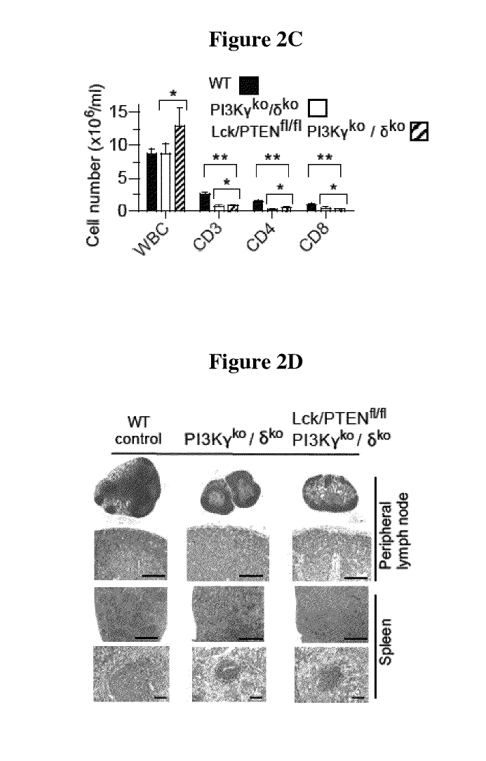

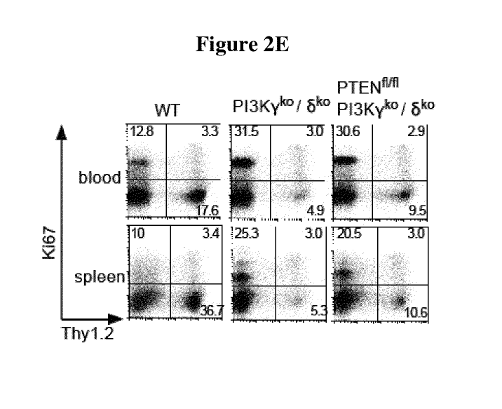

The role of PI3Kγ and PI3Kδ in tumorigenesis was further shown by the continued reduction in thymus size, cellularity, and disruption in corticomedullary differentiation in FIG. 2A-E. The absence of PTEN did not allow unrestricted regulation of PIP₃ of all class I PI3K isoforms in thymi of Lck/PTEN^{fl/fl} PI3Kγ^{ko}δ^{ko} mice. This was evidenced by the persistent diminution in the total number of CD4⁺CD8⁺ double positive thymocyte population and near basal levels of phosphorylated Akt/PKB (Ser473) as compared to mice deficient in PTEN alone. Cellular alterations associated with PI3K p110γ/δ double deficiency were also detected in the peripheral blood and secondary lymphoid organs of triple mutant mice and included a paucity of CD3⁺ T-cells. No active tumor was found in peripheral lymph nodes or spleen of these animals at about 7 months of age as determined by absence of staining for the proliferation marker Ki67 on Thy1.2 positive cells. The results suggest that PI3Kγ and PI3Kδ activities are involved in malignant transformation of T-cells.

### Example 4

### In vitro Potency and Specificity of Compound A

The potency and selectivity of Compound A was compared to other inhibitors having the PI3Kα-sparing activity, such as Compounds C and D. The structure of Compounds A, C and D are each showed below.

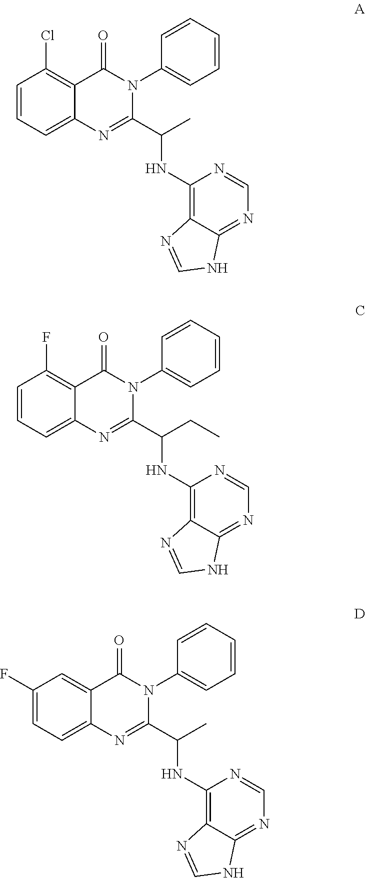 [SMILES: CC(Nc1ncnc2[nH]cnc12)c1nc2cccc(Cl)c2c(=O)n1-c1ccccc1 ; CCC(Nc1ncnc2[nH]cnc12)c1nc2cccc(F)c2c(=O)n1-c1ccccc1 ; CC(Nc1ncnc2[nH]cnc12)c1nc2ccc(F)cc2c(=O)n1-c1ccccc1]

In the examples herein, unless stated otherwise, Compounds C and D are the optically active form that predominantly includes the (S)-enantiomer. In this Example, both Compounds C and D were present in more than 99% enantiomeric excess.

All biochemical in vitro protein kinase assays presented in Table 4 were analyzed using the SelectScreen kinase inhibitor assay service (Invitrogen Ltd.). The potency of each compound was determined based on IC₅₀ data, and the selectivity of each compound was determined based on EC₅₀ data. Table 5 showed that Compound A had lower IC₅₀ and EC₅₀ values compared to those of Compounds C and D. This suggests that Compound A has the PI3Kα-sparing activity and that Compound A is more potent and selective compared to Compounds C and D.

TABLE 5
Comparison of IC₅₀ and EC₅₀ of Compounds A, C and D

| | Compound A | | Compound C | | Compound D | |
|---|---|---|---|---|---|---|
| | IC₅₀ | EC₅₀ | IC₅₀ | EC₅₀ | IC₅₀ | EC₅₀ |
| Isoform | (nM) | (nM) | (nM) | (nM) | (nM) | (nM) |
| p110α | 66 | 6,000 | 820 | >20,000 | 303 | >20,000 |
| p110β | 18 | 270 | 565 | 1,900 | 153 | 1,200 |
| p110γ | 36 | 677 | 85 | 3,000 | 25 | 2,345 |
| p110δ | 0.1 | 2.4 | 2.5 | 8.0 | 0.9 | 4.9 |

Effects of Compounds A, C, and D as PI3Kα-sparing inhibitors were further examined in other cellular assay.

Enzymatic activity of the class I PI3K isoforms was measured using a time resolved fluorescence resonance energy transfer assay (TR-FRET) that monitors formation of the product 3,4,5-inositol triphosphate molecule (PIP3), as it competes with fluorescently labeled PIP3 for binding to the GRP-1 pleckstrin homology domain protein. An increase in phosphatidylinositide 3-phosphate product results in a decrease in TR-FRET signal as the labeled fluorophore is displaced from the GRP-1 protein binding site. Class I PI3K isoforms were expressed and purified as heterodimeric recombinant proteins. All assay reagents and buffers for the TR-FRET assay were purchased from Millipore. PI3K isoforms were assayed under initial rate conditions in the presence of 25 mM Hepes (pH 7.4), and 2×Km ATP (100-300 μM), 10 μM PIP2, 5% glycerol, 5 mM MgCl2, 50 mM NaCl, 0.05% (v/v) Chaps, 1 mM dithiothreitol, 1% (v/v) DMSO at the following concentrations for each isoform: PI3Kα, β, δ at 50 picomolar (pM) and PI3Kγ at 2 nanomolar (nM). After an assay reaction time of 30 minutes at 25° C., reactions were terminated with a final concentration of 10 mM EDTA, 10 nM labeled-PIP3, and 35 nM Europium labeled GRP-1 detector protein before reading TR-FRET on an Envision plate reader. IC₅₀ values were calculated from the fit of the dose-response curves to a four-parameter equation. All IC₅₀ values represent geometric mean values of a minimum of four determinations. These assays generally produced results within 3-fold of the reported mean.

TABLE 6
IC₅₀ value of Compounds A, C, and D.

| | p110α | p110 β | p110δ | p110γ |
|---|---|---|---|---|
| Compound A | 2200 | 190 | 1.3 | 50 |
| Compound D | 7800 | 3400 | 9.3 | 1000 |
| Compound C | 10000 | 3200 | 14 | 1400 |

### Example 5

### Effect of Compounds in Solid Tumors, B-Cell Malignancies, and T-Cell Malignancies

Cellular viability and pAkt activity were used to determine effects of Compounds A, C, and D in solid tumor, B-cell malignancies, and T-cell malignancies. As shown in Table 7, Compound A was effective in T-cell malignancies, B-cell malignancies, and selected solid tumors having PTEN mutations. Compounds C and D were most effective against B cell malignancies and select solid tumors; however, they did not have similar levels of PI3Kα-sparing activity as Compound A. The results suggest that Compound A has a surprising PI3Kα-sparing activity against T-cell, B-cell, and other malignancies as compared to PI3Kα-sparing inhibitors Compounds C and D.

TABLE 7
Comparison of Viability and pAkt
activity of Compounds A, C and D

| | | Compound A | Compound C | Compound D |
|---|---|---|---|---|
| Indication | Read-out | EC₅₀ (nM) | EC₅₀ (nM) | EC₅₀ (nM) |
| Solid tumor | Viability | 1,700 | 11,400 | 4,750 |
| N = 14 | pAkt | 188 | ND | 987 |
| B Cell | Viability | 420 | 662 | 540 |
| Malignancies | pAkt | 184 | 367 | 350 |
| N = 7 | | | | |
| T Cell | Viability | 2,400 | >25,000 | 7,700 |
| Malignancies | pAkt | 310 | ND | 988 |
| N = 7 | | | | |

### Example 6

### Effect of Compound A in Solid Tumor Cell Lines

The effect of Compound A in CNS tumors, renal tumors, prostate tumors, melanoma tumors, ovarian tumors, breast tumors, colon tumors, and glioma tumors were determined. The results of cell proliferation, viability and apoptosis were summarized in Table 8. Compound A induced apoptosis in certain cell lines of prostate, ovarian, breast, and glioma tumors.

TABLE 8
Effect of Compound A in solid tumor cell lines

| | | pAkt EC₅₀ | | |
|---|---|---|---|---|
| | | (nM)/Pathway | Viability | Induction of |
| Cell Line | Tumor Type | inhibition | GI₅₀ (nM) | Apoptosis |
| SF-295 | CNS | 162 | ND | ND |
| 786-0 | Renal | 44 | 5,000 | ND |
| PC3 | Prostate | 155 | 2,400 | + |
| UACC-62 | Melanoma | 45 | ND | ND |
| IGROV-1 | Ovarian | 10 | 1,300 | ND |
| LNCaP | Prostate | 34 | 220 | + |
| OVCAR-3 | Ovarian | 647 | 850 | + |
| T47D | Breast | 502 | 1,700 | + |
| BT549 | Breast | 34 | ND | ND |
| KM-12 | Colon | 897 | ND | ND |
| RXF-393 | Renal | 25 | ND | ND |
| MDA-MB-468 | Breast | 56 | ND | ND |
| LN18 | Glioma | 10 | 3,500 | + |
| LN229 | Glioma | 10 | 3,000 | + |
| U87MG | Glioma | 17 | 2,500 | + |
| U138MG | Glioma | 2,290 | ND | ND |
| U251 | Glioma | 2,250 | ND | − |
| VCaP | Prostate | 1,200 | ND | + |
| 22Rv1 | Prostate | 250 | 2,300 | ND |

### Example 7

### Effect of Compound A on T-ALL

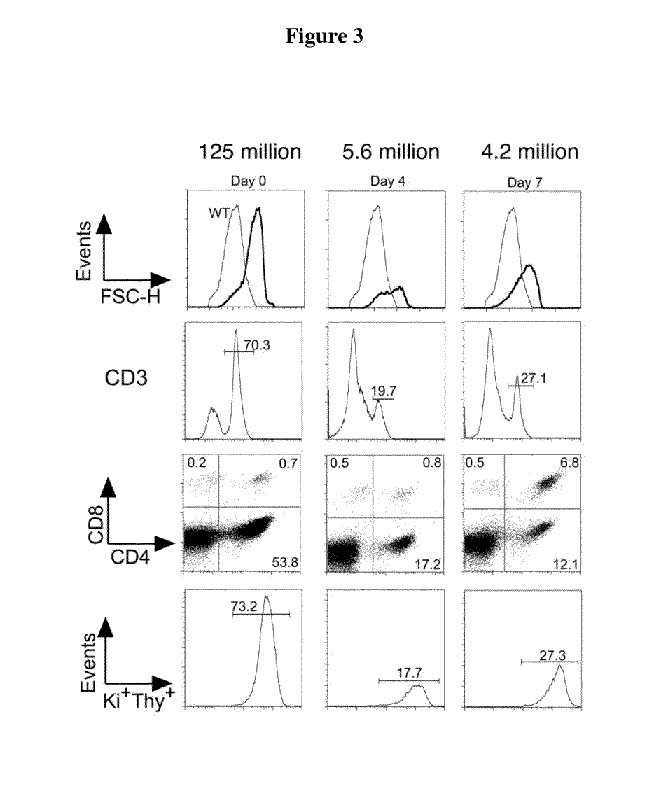

Lck/PTEN^{fl/fl} mice were crossed with the mice having a luciferase cDNA, preceded by a LoxP-stop-LoxP cassette was introduced into the ubiquitously expressed ROSA26 locus. The resulting mice were administered with Compound A at a dose of 30 mg/kg BID and examined for cell counts and luminescent signal at Day 0, 4, and 7. As shown in FIG. 3, in the presence of Compound A, the leukemia cells decreased from about 125 million at Day 0 to about 5.6 million at Day 4, and further decreased to about 4.2 million at Day 7. Also, the luminescent signal in mice treated with Compound A was significantly lower compared to those of the wild-type (WT) control mice at Day 4 and 7. The results are consistent with the reduction in whole blood cells and CD4 single positive population of tumor cells. Moreover, CD3 levels were reduced in mice administered with Compound A.

All animals showed a significant reduction of white blood cells by Day 4 reflected in the loss of the highly proliferative blast population (Thy1.2/Ki-67 double positive, high FSC-H). The blast population remained at low levels for the duration of treatment. The results suggest that Compound A reduces tumor burden in animals with PTEN null T-ALL.

### Example 8

### Anti-Leukemic Effects of Compounds B and E in PTEN Null T-ALL Tumors in Mice

Lck/PTEN^{fl/fl} mice having T-ALL were administered with Compound B at an oral dose of 10 mg/kg every 8 hours or DMSO vehicle for a period of 7 days. Candidate mice for the studies were ill-appearing, had a whole blood cell counts (WBC) above 45 K/μL, evidence of blasts on peripheral smear, and more than 75% of circulation cells staining double positive for Thy1.2 and Ki-67. Compound B extended the median survival to 45 days compared to 7.5 days for the vehicle group (data not shown).

Compound B can be synthesized as described in Sadhu, C. et al., Essential role of Phosphoinositide 3-kinase δ in neutrophil directional movement, J. Immunol. 170, 2647-2654 (2003). In the examples herein, unless stated otherwise, Compounds B and E are the optically active form that predominantly includes the (S)-enantiomer. The structure of Compounds B and E are each shown below.

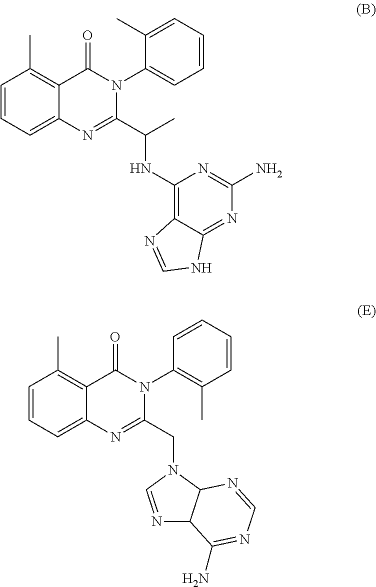 [SMILES: Cc1ccccc1-n1c(C(C)Nc2nc(N)nc3[nH]cnc23)nc2cccc(C)c2c1=O ; Cc1ccccc1-n1c(CN2C=NC3C(N)=NC=NC32)nc2cccc(C)c2c1=O]

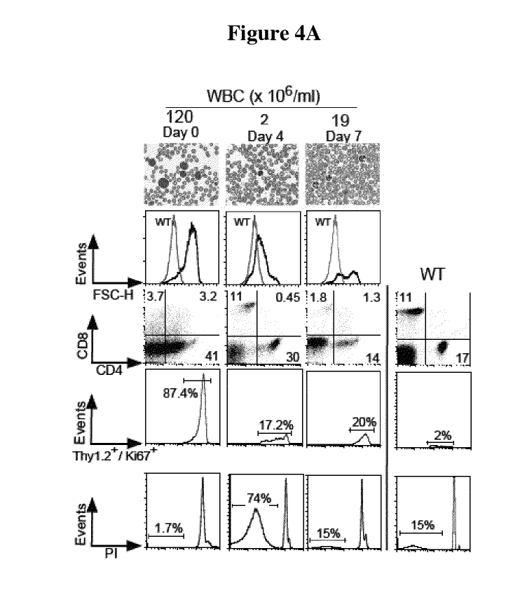

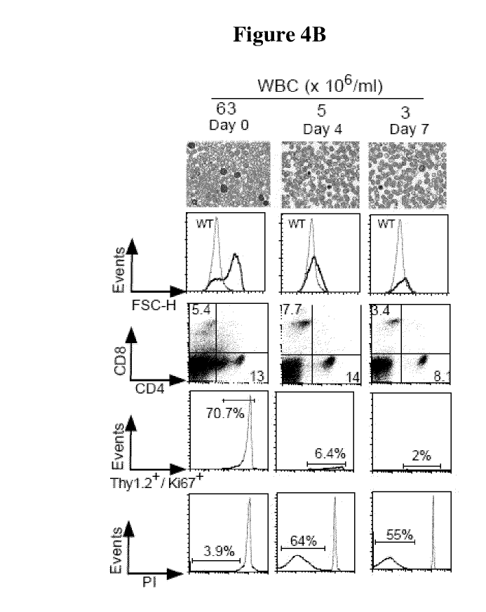

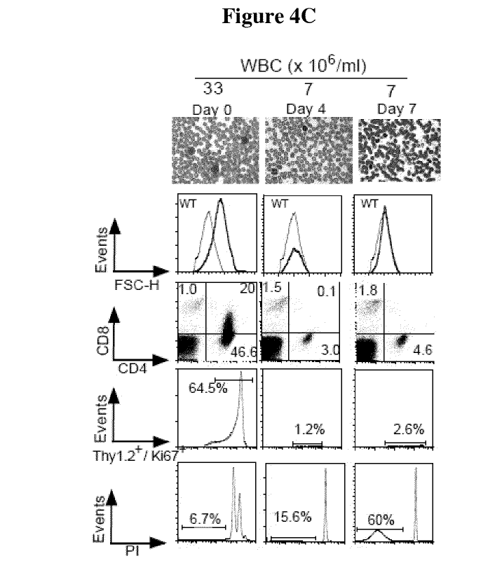

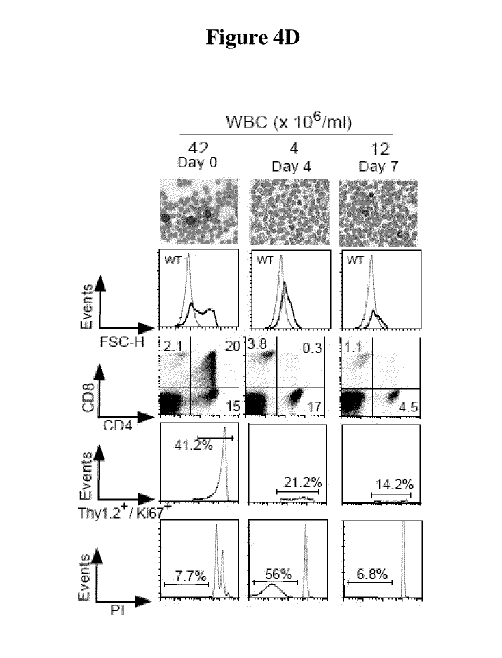

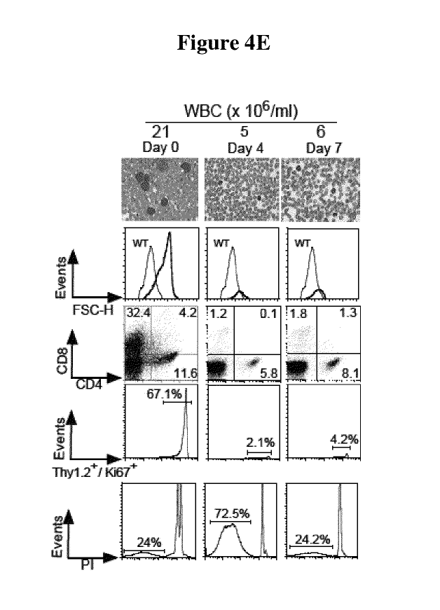

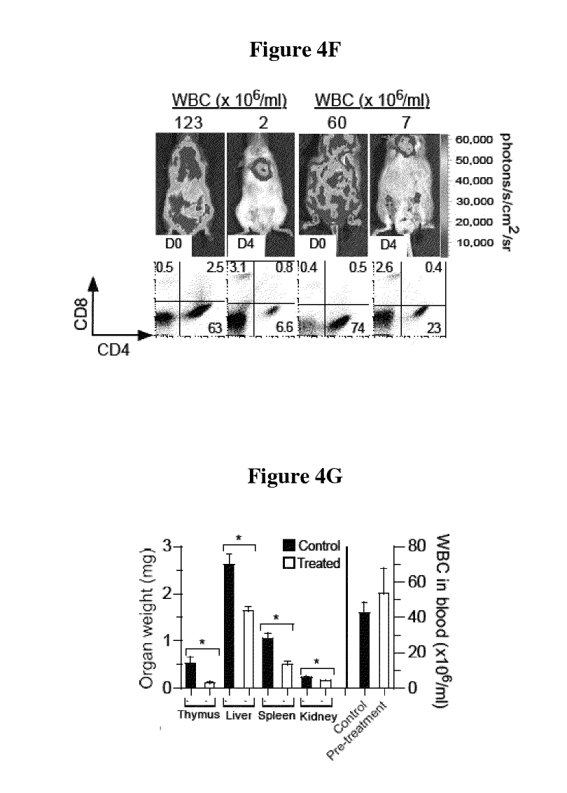

Diseased Lck/PTEN^{fl/fl} mice (i.e. Lck/PTEN^{fl/fl} mice diagnosed with T-ALL) were treated with Compounds B for a period of 7 days. Mice were examined for sequential blood counts, peripheral smears, and flow cytometric analyses. FIGS. 4A-D showed results of four different mice treated with Compound B. All animals showed a significant reduction in WBC by Day 4 reflected in the loss of the highly proliferative blast population (Thy1.2/Ki-67 double positive, high FSC-H), which remained at low levels for the duration of treatment. Moreover, both CD4 single positive and CD4/CD8 double positive T-ALL responded to Compound B, which corresponded with an increase in apoptosis detected as sub-G0 population after propidium iodide (PI) staining on Day 4 through Day 7. Forward scatter (FSC) and Ki67 staining were indicators of cell size and proliferation, respectively; and apoptosis was detected by assessing the sub-G0 population after PI staining.

Additionally, diseased Lck/PTEN^{fl/fl} PI3Kγ^{ko} mice were administered with a PI3Kδ selective inhibitor Compound E of 20 mg/kg. FIG. 4E showed results of Lck/PTEN^{fl/fl} PI3Kγ^{ko} mice treated with Compound E were similar to those of Lck/PTEN^{fl/fl} mice treated with Compound B. The results suggest that the reliance of PTEN null tumors on the combined activities of PI3Kγ and PI3Kδ.

Additional bioluminescent imaging showed effects of Compound B to reduce tumor burden. PTEN^{fl/fl} mice were crossed with the mice having a luciferase cDNA, preceded by a LoxP-stop-LoxP cassette was introduced into the ubiquitously expressed ROSA26 locus. Progeny were then crossed with Lck-cre transgenics to delete PTEN in T-cell progenitors and induce expression of luciferase. Imaging on T-ALL tumor bearing mice was performed at Day 0 and 4 after treatment of Compound B or vehicle. Signals at Day 4 were dramatically lower in treated animals, consistent with the reduction in the WBC count and the CD4 single positive population of tumor cells, as seen in FIG. 4F. Moreover, weights of thymi, liver, spleen, and kidneys from treated PTEN^{fl/fl} mice were significantly less than that for animals that received vehicle control for 7 days, as seen in FIG. 4G (P<0.01).

### Example 9

### Effects of Compounds B and E in CCRF-CEM Cells

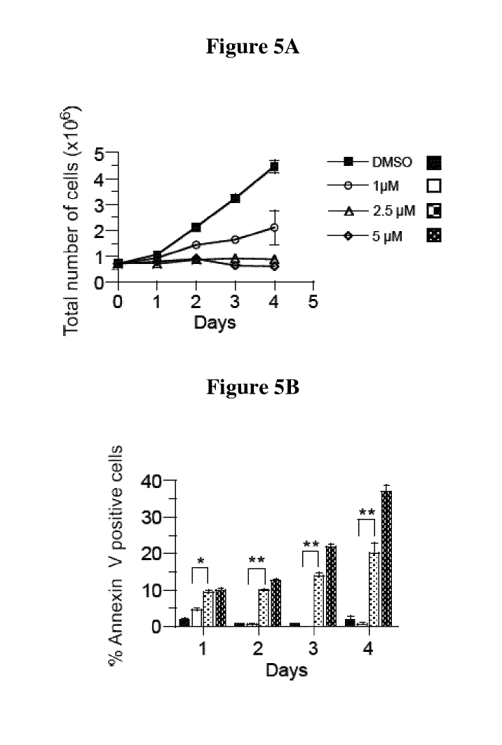

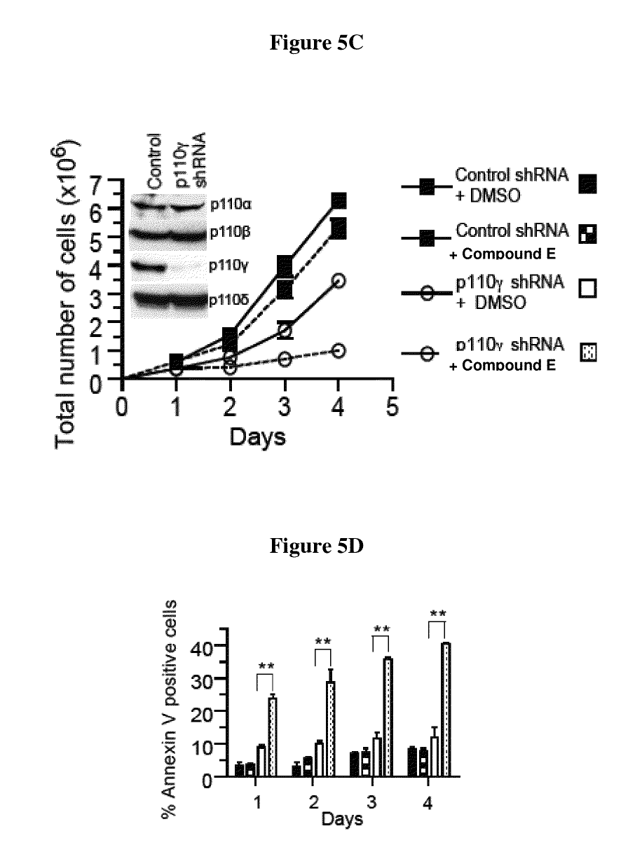

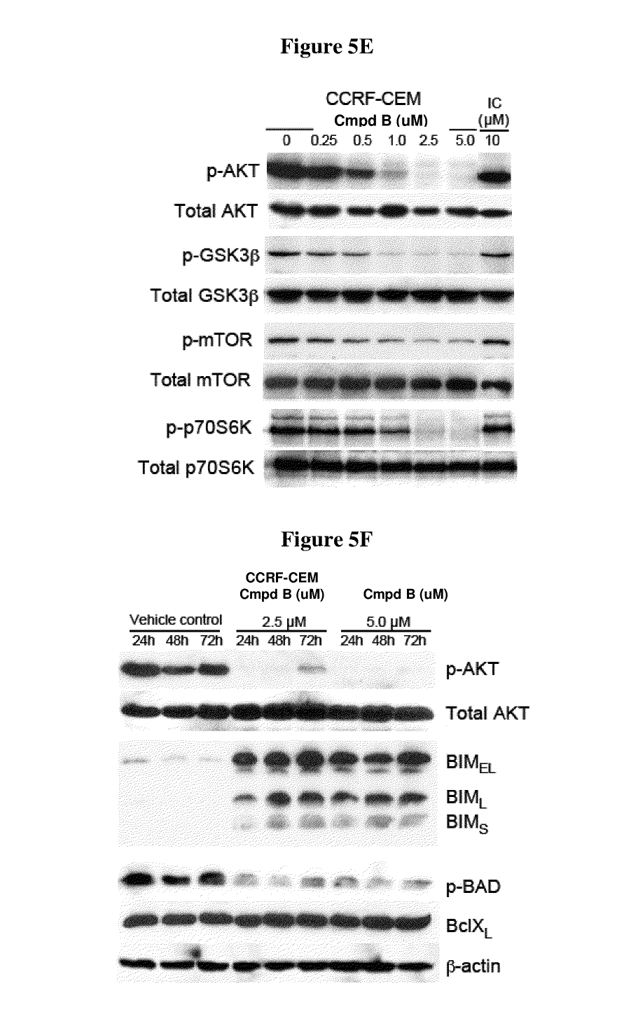

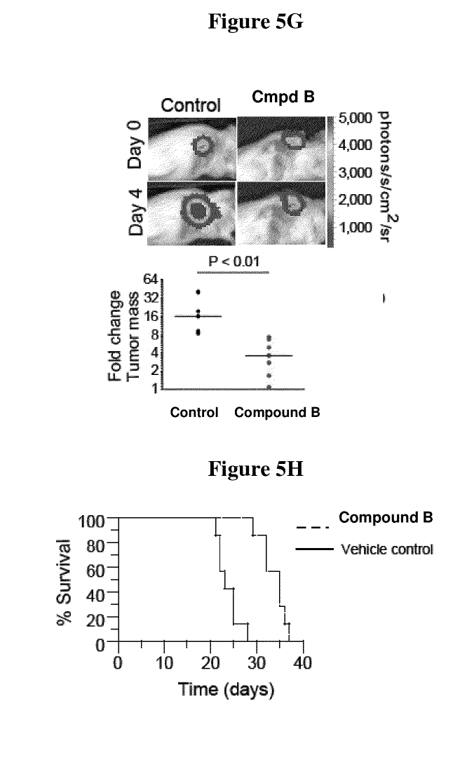

CCRF-CEM cells, a PTEN null acute lymphoblastoid leukemia cell line, were treated with Compound B of 1, 2.5, or 5 μM or control of DMSO vehicle for a period of 4 days. As shown in FIGS. 5A-B, Compound B prevented proliferation and promoted apoptosis within 24 hours, which persisted throughout the duration of 4 days. Increase in apoptosis represented a reduction in number of T-cells.

To demonstrate the importance of the combined activities of PI3Kγ and PI3Kδ for these processes in CCRF-CEM cells, a shRNA vector that targeted the p110γ catalytic domain was utilized. Immunoblot analysis revealed a>95% reduction in expression of p110γ with no affect on the other isoforms, as seen in FIG. 5C. Subsequent treatment of these cells with 10 μM of Compound E prevented proliferation and promoted apoptosis as observed for non-transfected CCRF-CEM exposed to Compound B, FIGS. 5C-D. In addition, Compound E had minimal effect on cells containing empty vector alone. Consistent with Example 7-8, the results suggests that PI3Kγ and PI3Kδ affect the proliferation and survival of T-ALL lymphoblasts.

The ability of Compound B treatment to interfere with proapoptotic effectors such as the BH3-only pro-apoptotic protein BAD and to repress the expression of BIM was also examined. CCRF-CEM cells were treated with Compound B ranging from 0, 0.25, 0.5, 1.0, 2.5, 5.0 and 10 μM. As shown in FIG. 5E, reduction and complete abrogation of Akt/PKB (Ser473) phosphorylation was detected in cells treated with 2.5 μM of Compound B. Downstream targets of this protein kinase were also observed to be affected as evidenced by the reduction in phosphorylation of GSK3β and mTOR. Consistent with the importance of PI3K in tumor cell survival, Compound B treatment resulted in a reduction in phosphorylation of BAD, as well as an enhanced expression of its counterpart BIM (including the L and S isoforms), as seen in FIG. 5F.

To assess the in vivo relevance of these observations, mice with subcutaneous or intravenous CCRF-CEM cells were treated with either Compound B or DMSO vehicle. In the subcutaneous xenographs, luciferase expressing CCRF-CEM cells were injected into the flanks of immunodeficient mice and allowed to grow for 1 week before administering vehicle control or 10 mg/kg of Compound B for a period of 4 days. In the intravenous xenographs, treatment commenced 3-day post-injection of tumor cells for a period of 7 days. Bioimaging of subcutaneous tumors revealed a 5-fold difference in luminescence in Compound B treated versus vehicle treated animals, as seen in FIG. 5G. This translated into an increase in median survival time for treated animals with systemic disease of 35 days versus 23 days for mice that received vehicle control alone, as seen in FIG. 5H(P<0.001). The results suggest that Compound B prevented the proliferation of CCRF-CEM cells implanted subcutaneously and increased the survival of NOD.Cg-Prkdc^{scid} Il2rg^{tmlWjl}/Sz that received these cells intravenously.

### Example 10

### Effects of Compounds A and B in T-ALL Tumors and Cells

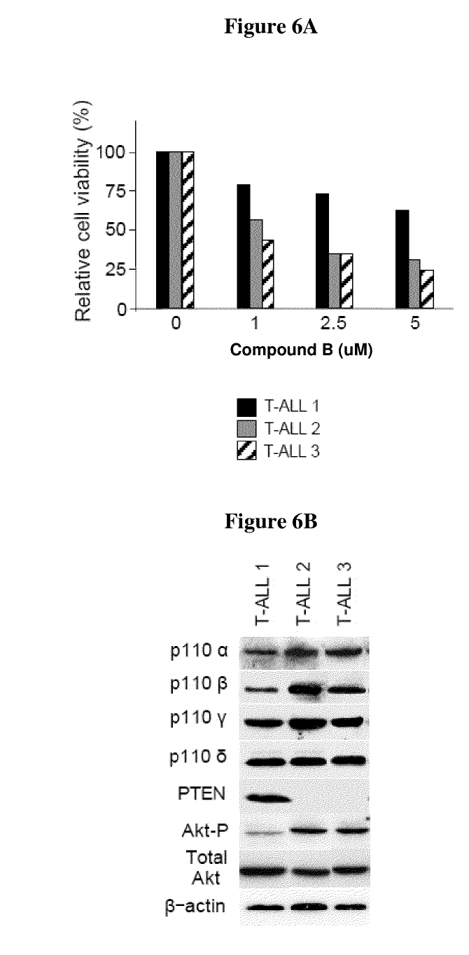

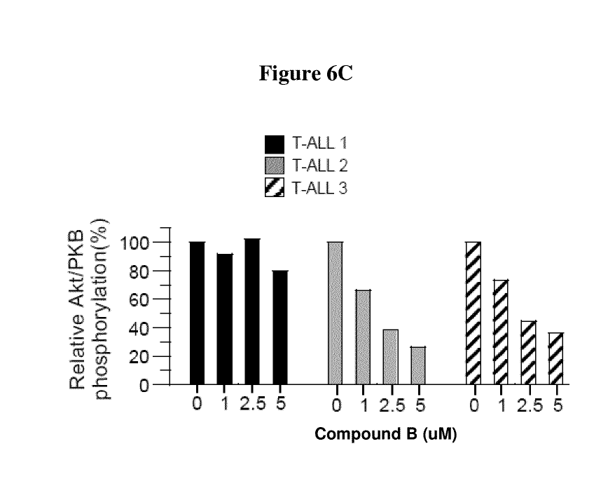

Primary T-ALL tumors isolated from three patients with active disease was treated with either Compound A or B, ranging from 0, 1.0, 2.5, and 5.0 μM, or DMSO vehicle. As shown in FIGS. 6A-B, the viability of tumor cells was reduced in the presence of Compound B and the lowest viability was in tumors devoid of PTEN. Also, the sensitivity or efficacy of Compound B was correlated with the level of inhibition to the Akt/PKB phosphorylation, as seen in FIG. 6C.

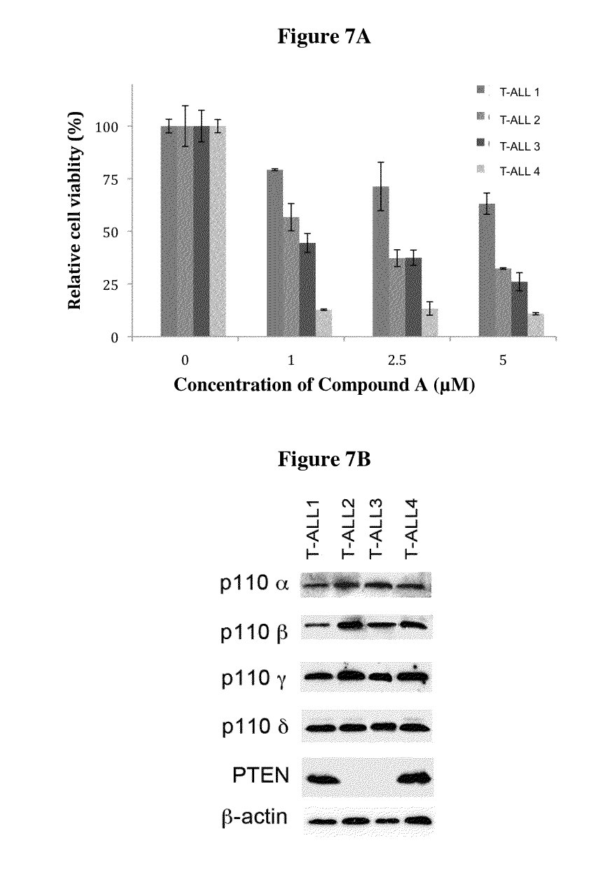

Effects of Compound A on the primary T-ALL tumors isolated from four patients with active disease were also determined. Similar to those of Compound B, treatment of Compound A results in the reduced viability of tumor cells as shown in FIG. 7A, and the inhibition to the Akt/PKB phosphorylation as shown in FIG. 7B. The results suggest that the sensitivity or efficacy of Compounds A and B is correlated with the phosphorylation state of Akt/PKB in primary T-ALL tumor cells.

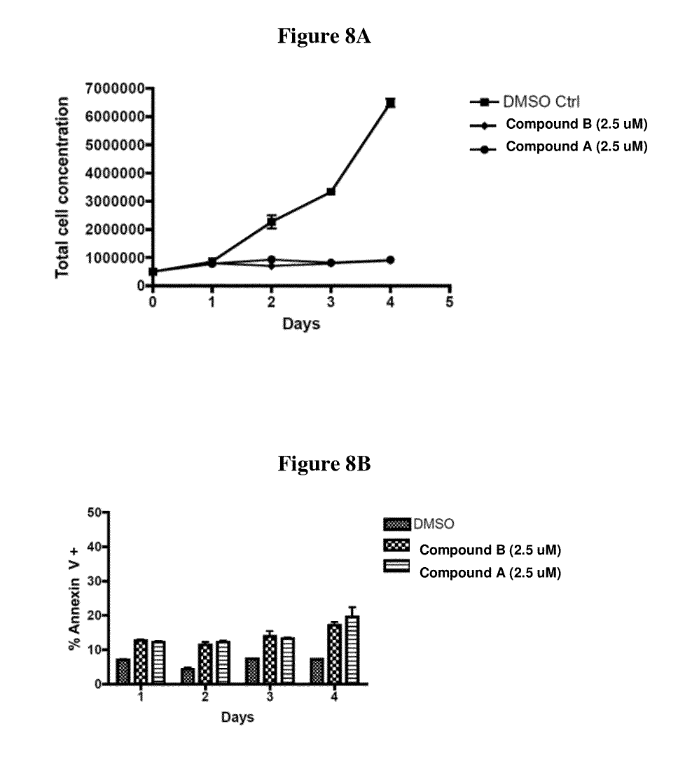

T-ALL cells were incubated with 2.5 μM of Compounds A or B, or DMSO vehicle for a period of 4 days. Within 24 hours, both Compounds A and B inhibited cell proliferation as shown in FIG. 8A. Also, both compounds induced apoptosis as shown by the reduction in of T-cells compared to the control of DMSO in FIG. 8B. This suggests that Compounds A and B are effective in treating T-ALL.

1. A compound having the structure of Compound A 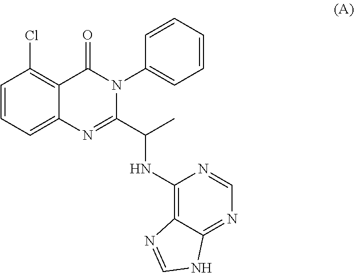 [SMILES: CC(Nc1ncnc2[nH]cnc12)c1nc2cccc(Cl)c2c(=O)n1-c1ccccc1]
or a pharmaceutically acceptable salt thereof.

2. The compound according to claim 1 or a pharmaceutically acceptable salt thereof, wherein the compound or a pharmaceutically acceptable salt thereof is the (S)-enantiomer.

3. A composition comprising the compound according to claim 1 or a pharmaceutically acceptable salt thereof, and at least one pharmaceutically acceptable excipient.

4. The composition according to claim 3, wherein the composition comprises the (S)-enantiomer of the compound or a pharmaceutically acceptable salt thereof,
wherein the (S)-enantiomer of the compound or a pharmaceutically acceptable salt thereof is present in excess of the (R)-enantiomer of the compound or a pharmaceutically acceptable salt thereof.

5. The composition according to claim 4, wherein the composition is substantially free of the (R)-enantiomer of the compound or a pharmaceutically acceptable salt thereof.

6. A method of treating a condition in a patient, wherein the condition is cancer, comprising administering to the patient a composition comprising the compound according to claim 1 and at least one pharmaceutically acceptable excipient.

7. The method according to claim 6, wherein the composition comprises the (S)-enantiomer of the compound or a pharmaceutically acceptable salt thereof,
wherein the (S)-enantiomer of the compound or a pharmaceutically acceptable salt thereof is present in excess of the (R)-enantiomer of the compound or a pharmaceutically acceptable salt thereof.

8. The method according to claim 7, wherein the composition is substantially free of the (R)-enantiomer of the compound or a pharmaceutically acceptable salt thereof.

9. The method according to claim 7, wherein the (S)-enantiomer of the compound or a pharmaceutically acceptable salt thereof predominates over the (R)-enantiomer of the compound or a pharmaceutically acceptable salt thereof by a ratio of at least 9:1.

10. The method according to claim 6, wherein cancer is a hematologic malignancy.

11. The method according to claim 10, wherein the hematologic malignancy is leukemia or lymphoma.

12. The method according to claim 10, wherein the hematologic malignancy is selected from the group consisting of acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma, Waldestrom's macroglobulinemia (WM), T-cell lymphoma, B-cell lymphoma, and diffuse large B-cell lymphoma (DLBCL).

13. The method according to claim 6, wherein the cancer is a solid tumor.

14. The method according to claim 13, wherein the solid tumor is selected from the group consisting of pancreatic cancer, bladder cancer, colorectal cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancers, CNS cancers, brain tumors, bone cancer, soft tissue sarcoma, non-small cell lung cancer, small-cell lung cancer, and colon cancer.

15. The method according to claim 6, wherein the patient is refractory to chemotherapy treatment or in relapse after treatment with chemotherapy.

16. The method according to claim 6, wherein the compound or a pharmaceutically acceptable salt thereof is administered at a dose of about 1-4,000 mg/day.

17. The method according to claim 6, further comprising reducing the level of PI3Kδ, PI3Kγ, or PI3Kβ activity in the patient.

18. The method according to claim 6, further comprising administering to the patient, in addition to the compound or a pharmaceutically acceptable salt thereof, a therapeutically effective amount of at least one therapeutic agent selected to treat the cancer in the patient.

19. The method according to claim 18, wherein the therapeutic agent is selected from the following group consisting of Bortezomib (VELCADE®), Carfilzomib (PR-171), PR-047, disulfuram, lactacystin, PS-519, eponemycin, epoxomycin, aclacinomycin, CEP-1612, MG-132, CVT-63417, PS-341, vinyl sulfone tripeptide inhibitors, ritonavir, PI-083, (+/−)-7-methylomuralide, (−)-7-methylomuralide, Perifosine, Rituximab, Sildenafil citrate (VIAGRA®), CC-5103, Thalidomide, Epratuzumab (hLL2-anti-CD22 humanized antibody), Simvastatin, Enzastaurin, Campath-1H, Dexamethasone, DT PACE, oblimersen, antineoplaston A10, antineoplason AS2-1, alemtuzumab, beta alethine, cyclophosphamide, doxorubicin hydrochloride, PEGylated liposomal doxorubicin hydrochloride, prednisone, prednisolone, cladribine, vincristine sulfate, fludarabine, filgrastim, melphalan, recombinant interferon alfa, carmustine, cisplatin, cyclophosphamide, cytarabine, etoposide, melphalan, dolastatin 10, indium In 111 monoclonal antibody MN-14, yttrium Y 90 humanized epratuzumab, anti-thymocyte globulin, busulfan, cyclosporine, methotrexate, mycophenolate mofetil, therapeutic allogeneic lymphocytes, Yttrium Y 90 ibritumomab tiuxetan, sirolimus, tacrolimus, carboplatin, thiotepa, paclitaxel, aldesleukin, recombinant interferon alfa, docetaxel, ifosfamide, mesna, recombinant interleukin-12, recombinant interleukin-11, Bcl-2 family protein inhibitor ABT-263, denileukin diftitox, tanespimycin, everolimus, pegfilgrastim, vorinostat, alvocidib, recombinant flt3 ligand, recombinant human thrombopoietin, lymphokine-activated killer cells, amifostine trihydrate, aminocamptothecin, irinotecan hydrochloride, caspofungin acetate, clofarabine, epoetin alfa, nelarabine, pentostatin, sargramostim, vinorelbine ditartrate, WT-1 analog peptide vaccine, WT1 126-134 peptide vaccine, fenretinide, ixabepilone, oxaliplatin, monoclonal antibody CD19, monoclonal antibody CD20, omega-3 fatty acids, mitoxantrone hydrochloride, octreotide acetate, tositumomab and iodine I131 tositumomab, motexafin gadolinium, arsenic trioxide, tipifarnib, autologous human tumor-derived HSPPC-96, veltuzumab, bryostatin 1, anti-CD20 monoclonal antibodies, chlorambucil, pentostatin, lumiliximab, apolizumab, Anti-CD40, ofatumumab, bendamustine, and a combination thereof.

20. A kit comprising the compound according to claim 1 or a pharmaceutically acceptable salt thereof.
